SEQUENCE LISTING (69 sequences)

SEQ ID NO: 1 - DNA, 2996 bp, Bos taurus
  CDS (147)..(1031)
ggcacgaggtctgctgccgctccctctgcccagtccggccgggcagcgggcacgagcagacccaacagcagctccaagcgcggtgcccgaggccaggctctgcttcgtcctcgagcccctctgagtgaacactggctgtcgccatcatgtctgctgcaaatcctgagactccaaactcaaccatctccagagaggccagcacccagtcgtcttcagctaccaccagccaaggctatgttttaccagaaggcaaaatcatgccaaacactgtttttgtcggtggaattgatgttaggatggatgaaacagaaattagaagtttctttgctagatatggttcagtaaaagaagtgaagataatcacggatcgaactggtgtgtccaaaggctatggatttgtttcattttataatgacgtggatgtgcagaagatagtagaatcacagataaatttccatggtaaaaagctgaaactgggccctgcaatcaggaaacaaaatttgtgtgcttatcatgtgcagccacgtcctttggtttttaatcctcctcctccaccacaatttcagagtgtctggagtaatccaaacgctgaaacttacatgcagcctccaaccatgataaaccctataactcagtatgttcaggcttatcctccttatccaagttcaccagttcaggttatcactggatatcagctgcctgtttataattatcagatgccaccgcagtggcctgctggggaacaaaggagttatgttattcctccggcttatacaaccattaactaccactgtaatgaagttgacaccggagctgacgttttgcccagtgaatgctcggttcatgaagctactccgtcctctggaaatggcccacaaaagaaatctgtggacagaagcatacagacggtggtatcttgtctatttaatccagagaatagactgagaaactctgttgtcactcaagatgactacttcaaggataaaagagttcatcactttagaagaagtcgggcagtgcttaagtctgtttgatcctctctgctaactttctagtctcataggaagtcacaggttttgaatagtaagagactgaaagttttccactgttatagaaatttaattctgaattttgataaatcacactcagactttctatatgttatattagattgcttagttttattttacattgaaactttttcattagacatttacttagaaactggttctgtgttgaatatatagttaaaagttaaaaaataattgagactgaaaagaataactttaagatatatctgtaaggatttttacctgacattttaagagtttaaaaacaaatgctgattttttgggaaaaaaaaattgttttagaaaacctattttgaaaggtcagaattttgatagtttaaatacaagcacttcacttctccaacaagtacctatgaacagtacagtatctgcaatattgtgcttttatgatttgtctagaaatttaccacaaaagcagagtttttaaaactgaatttttaatcagtgaaactcaatgtatagttagctttattgaagttttccttatctaaacccagtaaaacagattctaaacaaacagtccaatcagtgggtcttatgtttattacttcaaaatactttatcttttatctagaatccacacatagaggtatctgatgggatggtaattaggattactaaaattctgggccaaatttttttttaaaagaatctaaaacaaacttaacttttctaggtacataaacttcttagttgagttaccattcttttttcttttactttttttttccccttgaaatgttaaactcaatggctgtattttaaattgtgcaaaatattggtattaaagaaggctgaaactttgtcacttaaaagttaatcagagtatctgaaaggaattgtttttataagaaacattaaaatattagctatttgttaaagaacagatagattttaatttttttttagcctgttatttttctttcttttttttttttttaagtaaaatatgtccaggagagtcagagtagttttgatgtttctaaaccacaaaagttgtttagtaataagcatatccccaaaacacacttgagttagttaacagtccatttttagatgaggtggctcctcctcagacattgtaaatcgtgacaagcacctggagatccagagcccctttgctgtacccacagtcttgcttctccagtctccactgttcctgcaggtgctcctggaacagcgatggaaaggaaaggaggaggagtataagcagtttgagggttaagccttggaaggaaagggtagcagtgggataatccctttctaagggattcacttggagcagtatcgtttgacctgccatgggtttagaggagcttcctggcttttctgtcttgggccagccccttcattaaaacacggaaactccgtatagccctgtcctgacgaggcttccgtgggccgtgccatcagtgcccgttgtcttagcactcctaataattttcgttctgaatctgaagggaagaatcttttaacttaatgatacccaagagggcttttttatgcctcagaatttgaaagtgccctgaaatttgttctttttaaaattctctgtacttttttgtgtgtgtgctataacattcttacatgtattattctgtgattaaatctccaggttactaaaaatgatacctaaattctaaagagccaattctcattattccagtatgaccttattcccttaagaaaaagggaataaacattttgtctgctattttgttggactgaagtatttaaaagagtaaggtagaagagacaaaatcctgaacctttcaaaatggaaaattaattttaaacttaaaaatgtgttcctattacctatgctgatactgtctttgcataaatgaataaatagaaataaagaaaaaaaaaaaaaaaaaaaaaaaaaaaaaaaaaaaaaaaaaa

SEQ ID NO: 2 - protein, 295 aa, Bos taurus
MSAANPETPNSTISREASTQSSSATTSQGYVLPEGKIMPNTVFVGGIDVRMDETEIRSFFARYGSVKEVKIITDRTGVSKGYGFVSFYNDVDVQKIVESQINFHGKKLKLGPAIRKQNLCAYHVQPRPLVFNPPPPPQFQSVWSNPNAETYMQPPTMINPITQYVQAYPPYPSSPVQVITGYQLPVYNYQMPPQWPAGEQRSYVIPPAYTTINYHCNEVDTGADVLPSECSVHEATPSSGNGPQKKSVDRSIQTVVSCLFNPENRLRNSVVTQDDYFKDKRVHHFRRSRAVLKSV

SEQ ID NO: 3 - DNA, 10 bp, Bos taurus
aacaataact

SEQ ID NO: 4 - DNA, 20 bp, Artificial Sequence
  Synthetic construct  oliognucleotide useful as a primer.
gttcctcttcaccttcttgg

SEQ ID NO: 5 - DNA, 21 bp, Artificial Sequence
  Synthetic construct  oligonucleotide useful as a primer.
ccttatgtttacaacactacc

SEQ ID NO: 6 - DNA, 20 bp, Artificial Sequence
  Synthetic construct  oligonucleotide useful as a primer.
ggctatgttttaccagaagg

SEQ ID NO: 7 - DNA, 18 bp, Artificial Sequence
  Synthetic construct  oligonucleotide useful as a primer.
gacacaccagttcgatcc

SEQ ID NO: 8 - DNA, 20 bp, Artificial Sequence
  Synthetic construct  oligonucleotide useful as a primer.
gattaaggcaatgtaggaac

SEQ ID NO: 9 - DNA, 20 bp, Artificial Sequence
  Synthetic construct  oligonucleotide useful as a primer.
atcttctgcacatccacgtc

SEQ ID NO: 10 - DNA, 20 bp, Artificial Sequence
  Synthetic construct  oligonucleotide useful as a primer.
catgtactgatacggtggtg

SEQ ID NO: 11 - DNA, 20 bp, Artificial Sequence
  Synthetic construct  oligonucleotide useful as a primer.
agtgaaggtaagtaaggtgg

SEQ ID NO: 12 - DNA, 20 bp, Artificial Sequence
  Synthetic construct  oliognucleotide useful as primer.
ttatcatggttggaggctgc

SEQ ID NO: 13 - DNA, 20 bp, Artificial Sequence
  Synthetic construct  oligonucleotide useful as a primer.
attgcagggcccagtttcag

SEQ ID NO: 14 - DNA, 20 bp, Artificial Sequence
  Synthetic construct  oligonucleotide useful as a primer.
ggaattgttcttgttgacac

SEQ ID NO: 15 - DNA, 20 bp, Artificial Sequence
  Synthetic construct  oligonucleotide useful as a primer.
acagcactaggattgtcacg

SEQ ID NO: 16 - DNA, 19 bp, Artificial Sequence
  Synthetic construct  oligonucleotide useful as a primer.
catgaaagcactcactcag

SEQ ID NO: 17 - DNA, 20 bp, Artificial Sequence
  Synthetic construct  oligonucleotide useful as a primer.
acagtgtcacagatacggtc

SEQ ID NO: 18 - DNA, 20 bp, Artificial Sequence
  Synthetic construct  oligonucleotide useful as a primer.
tgaccgtatctgtgacactg

SEQ ID NO: 19 - DNA, 20 bp, Artificial Sequence
  Synthetic construct  oliognucleotide useful as a primer.
caggcatacttgatatggca

SEQ ID NO: 20 - DNA, 19 bp, Artificial Sequence
  Synthetic construct  oligonucleotide useful as a primer.
gaacctcagaacaatacac

SEQ ID NO: 21 - DNA, 19 bp, Artificial Sequence
  Synthetic construct  oligonucleotide useful as a primer.
gacagaagccatactaatc

SEQ ID NO: 22 - DNA, 20 bp, Artificial Sequence
  Synthetic construct  oligonucleotide useful as a primer.
acatactatgtgacatggtg

SEQ ID NO: 23 - DNA, 20 bp, Artificial Sequence
  Synthetic construct  oligonucleotide useful as a primer.
ctcactctcaggctacagtc

SEQ ID NO: 24 - DNA, 19 bp, Artificial Sequence
  Synthetic construct  oligonucleotide useful as a primer.
gtctcttaggtcataatgg

SEQ ID NO: 25 - DNA, 20 bp, Artificial Sequence
  Synthetic construct  oligonucleotide useful as a primer.
aactactctgactctcctgg

SEQ ID NO: 26 - DNA, 20 bp, Artificial Sequence
  Synthetic construct  oligonucleotide useful as a primer.
ctgaatttcaaggatctcac

SEQ ID NO: 27 - DNA, 20 bp, Artificial Sequence
  Synthetic construct  oligonucleotide useful as a primer.
actgcttatactcctcctcc

SEQ ID NO: 28 - DNA, 20 bp, Artificial Sequence
  Synthetic construct  oligonucleotide useful as a primer.
aactggttctgtgttgaata

SEQ ID NO: 29 - DNA, 20 bp, Artificial Sequence
  Synthetic construct  oligonucleotide useful as a primer.
gaaggctgaaactttgtcac

SEQ ID NO: 30 - DNA, 20 bp, Artificial Sequence
  Synthetic construct  oligonucleotide useful as a primer.
atcgtgacaagcacctggag

SEQ ID NO: 31 - DNA, 20 bp, Artificial Sequence
  Synthetic construct  oligonucleotide useful as a primer.
gtctcataggaagtcacagg

SEQ ID NO: 32 - DNA, 20 bp, Artificial Sequence
  Synthetic construct  oligonucleotide useful as a primer.
tacaagcacttcacttctcc

SEQ ID NO: 33 - DNA, 10 bp, Bos taurus
agaggtaact

SEQ ID NO: 34 - DNA, 10 bp, Bos taurus
ggaggtaagt

SEQ ID NO: 35 - DNA, 10 bp, Bos taurus
aacggctacc

SEQ ID NO: 36 - DNA, 10 bp, Bos taurus
agaggtagct

SEQ ID NO: 37 - DNA, 10 bp, Bos taurus
agagatagct

SEQ ID NO: 38 - DNA, 10 bp, Bos taurus
agaagtaact

SEQ ID NO: 39 - DNA, 10 bp, Bos taurus
aacgactacc

SEQ ID NO: 40 - DNA, 10 bp, Bos taurus
aacgataact

SEQ ID NO: 41 - DNA, 10 bp, Bos taurus
agaaatagct

SEQ ID NO: 42 - DNA, 10 bp, Bos taurus
ggaggtaggt

SEQ ID NO: 43 - DNA, 10 bp, Bos taurus
agagataact

SEQ ID NO: 44 - DNA, 10 bp, Bos taurus
ggagataggt

SEQ ID NO: 45 - DNA, 20 bp, Artificial Sequence
  Synthetic construct  oligonucleotide useful as a primer.
ctcaaccatctccagagagg

SEQ ID NO: 46 - DNA, 20 bp, Artificial Sequence
  Synthetic construct  oligonucleotide useful as a primer.
ctagcatcaattccaccaac

SEQ ID NO: 47 - DNA, 18 bp, Artificial Sequence
  Synthetic construct  oligonucleotide useful as a primer.
gagcagacccaacagcag

SEQ ID NO: 48 - DNA, 18 bp, Artificial Sequence
  Synthetic construct  oligonucleotide useful as a primer.
ctgctgttgggtctgctc

SEQ ID NO: 49 - DNA, 18 bp, Artificial Sequence
  Synthetic construct  oligonucleotide useful as a primer.
cctctgagtgaacactgg

SEQ ID NO: 50 - DNA, 19 bp, Artificial Sequence
  Synthetic construct  oligonucleotide useful as a primer.
ctgtcgccatcatgtctgc

SEQ ID NO: 51 - DNA, 20 bp, Artificial Sequence
  Synthetic construct  oligonucleotide useful as a primer.
ggctatgttttaccagaagg

SEQ ID NO: 52 - DNA, 20 bp, Artificial Sequence
  Synthetic construct  oligonucleotide useful as a primer.
ccttctggtaaaacatagcc

SEQ ID NO: 53 - DNA, 18 bp, Artificial Sequence
  Synthetic construct  oligonucleotide useful as a primer.
gacacaccagttcgatcc

SEQ ID NO: 54 - DNA, 20 bp, Artificial Sequence
  Synthetic construct  oligonucleotide useful as a primer.
cgtggatgtgcagaagatag

SEQ ID NO: 55 - DNA, 20 bp, Artificial Sequence
  Synthetic construct  oligonucleotide useful as a primer.
atcttctgcacatccacgtc

SEQ ID NO: 56 - DNA, 20 bp, Artificial Sequence
  Synthetic construct  oligonucleotide useful as a primer.
attgcagggcccagtttcag

SEQ ID NO: 57 - DNA, 20 bp, Artificial Sequence
  Synthetic construct  oligonucleotide useful as a primer.
gcagcctccaaccatgataa

SEQ ID NO: 58 - DNA, 20 bp, Artificial Sequence
  Synthetic construct  oligonucleotide useful as a primer.
ttatcatggttggaggctgc

SEQ ID NO: 59 - DNA, 24 bp, Artificial Sequence
  Synthetic construct  oligonucleotide useful as a primer.
ggtagaagagacaaaatcctgaac

SEQ ID NO: 60 - DNA, 26 bp, Artificial Sequence
  Synthetic construct  oligonucleotide useful as a primer.
ttaatccagagaatagactgagaaac

SEQ ID NO: 61 - DNA, 26 bp, Artificial Sequence
  Synthetic construct  oligonucleotide useful as a primer.
gtttctcagtctattctctggattaa

SEQ ID NO: 62 - DNA, 20 bp, Artificial Sequence
  Synthetic construct  oligonucleotide useful as a primer.
agtcgggcagtgcttaagtc

SEQ ID NO: 63 - DNA, 20 bp, Artificial Sequence
  Synthetic construct  oligonucleotide useful as a primer.
gtctcataggaagtcacagg

SEQ ID NO: 64 - DNA, 20 bp, Artificial Sequence
  Synthetic construct  oligonucleotide useful as a primer.
cctgtgacttcctatgagac

SEQ ID NO: 65 - DNA, 24 bp, Artificial Sequence
  Synthetic construct  oligonucleotide useful as a primer.
ggtagaagagacaaaatcctgaac

SEQ ID NO: 66 - DNA, 24 bp, Artificial Sequence
  Synthetic construct oligonucleotide useful as a primer.
gttcaggattttgtctcttctacc

SEQ ID NO: 67 - DNA, 22 bp, Artificial Sequence
  Synthetic construct  oligonucleotide useful as a primer.
gcaaagacagtatcagcatagg

SEQ ID NO: 68 - DNA, 27 bp, Artificial Sequence
  Synthetic construct  oligonucleotide useful as a primer.
gtgacactcactcttctacctttgatc

SEQ ID NO: 69 - DNA, 19 bp, Artificial Sequence
  Synthetic construct  oligonucleotide useful as a primer.
ggtccaccaccctgttgct

(19) United States
(12) Patent Application Publication
(10) Pub. No.: US 2010/0009374 A1
(43) Pub. Date: Jan. 14, 2010

(54) SIRE EARLY SELECTION FOR MALE FERTILITY USING SINGLE NUCLEOTIDE POLYMORPHISMS (SNPS) OF THE DAZL GENE

(76) Inventors: Wansheng Liu, State College, PA (US); Aihua Wang, Blacksburg, VA (US)

Correspondence Address: UNR/DRI Technology Transfer Office, UNR-DRI Technology Transfer Office, Mail stop 321, 1664 N. Virginia St, Reno, NV, 89557, US

(21) Appl. No.: 12/522,916

(22) PCT Filed: Jan. 11, 2008
(86) PCT No.: PCT/US08/50937

§ 371 Date: Aug. 5, 2009

Related U.S. Application Data
(60) Provisional application No. 60/884,844, filed on Jan. 12, 2007.

(51) Int. Cl. C12Q 1/68

(52) U.S. Cl. 435/6

(57) ABSTRACT

Methods and materials for identifying bovine males which will produce semen which will exhibit a higher rate of successfully impregnation by either artificial insemination or natural mating. The method employs SNPs that have been identified in the bDAZL gene which are associated with enhanced (or decreased) male fertility in bovine males and haplotypes formed from such SNPs. The method herein can be used to identify male dairy or beef cattle. The method herein can be applied to bovine animals at an appropriate time and particularly at birth or in utero. The invention further provides kits for conduction assays to assess bovine male fertility/infertility. The invention additionally provides a method of breeding cattle employing the methods and materials herein.

### CROSS-REFERENCE TO RELATED APPLICATIONS

This application claims the benefit of U.S. provisional application Ser. No. 60/884,844, filed Jan. 12, 2007 which is incorporated by reference herein in its entirety.

### STATEMENT REGARDING UNITED STATES GOVERNMENT SUPPORT

This invention was made with funding from the United States Government, through the USDA, CSREES grant No. 2005-35205-15455. The United States Government has certain rights in this invention.

### BACKGROUND OF THE INVENTION

Infertility or subfertility is a common problem in mammals including human, mouse, cattle and other farm animals. Approximately 10-15% of couples world-wide are affected by reduced rates of fertility and about half of these cases result from the absence of sperm production in males. Cytological analysis of patients with azoospermia identified a deletion on the Y chromosome in a significant proportion of cases implying a genetic component to the phenotype. This region was proposed to contain an Azoospermia Factor (AZF). Subsequent analysis of this region identified three sub-regions, termed AZFa, AZFb and AZFc. Deleted in Azoospermia (DAZ) is one of the nine testis-specific gene families in the AZFc region. The DAZ gene family consists of three genes: BOLL [bol, boule-like (Burgoyne)], DAZ and DAZL (DAZ-Like) (Agulnik et al., 1998). It is believed that the ancestor member of the family is BOLL, which gave rise to DAZL via duplication prior to the divergence of vertebrates and invertebrates (Cauffman et al., 2005). Later, during primate evolution, the autosomal DAZL gene gave rise to DAZ on the Y chromosome by transposition, repeat amplification and pruning (Gromoll et al., 1999; Saxena et al., 1996; Shan et al., 1996). As a result, DAZ has been identified only in Old World monkeys and great apes, while DAZL is present in all vertebrates (Cooke et al., 1996; Saxena et al., 1996).

Yet, very few genes that are associated with measures of germ cell defects in humans have been identified (Tung et al. 2006). The DAZ family is expressed exclusively in the germ cells and encodes proteins that contain a highly conserved RNA-recognition motif (RRM) and a unique DAZ repeat of 24 amino acid residues. Both DAZ and DAZL are detected in the nuclei of primordial germ cells (PGCs) in fetal gonads (Xu et al., 2001), and are believed to function in the development of PGCs and in germ cell differentiation and maturation.

Mutations in these genes have been linked to infertility in several species including human, mouse, fly, and frog (Eberhart et al., 1996; Houston and King, 2000; Ruggiu et al., 1997). The infertile phenotype of Dazl null mouse (Dazl−/−) can be partially rescued by a human DAZ transgene (Slee et al., 1999). As the gene name suggests, deletion or microdeletion in DAZ gene(s) on the Y chromosome leads to severe spermatogenic failure and infertility in men (Ferlin et al., 1999; Reijo et al., 1995), and DAZL transcripts in the testes are lower in men with spermatogenic failure compared with fertile men (Lin et al., 2001).

A single nucleotide polymorphism (SNP) (A/G transition, T54A (Thr₅₄→Ala in exon 3) in the RNA-binding domain of the DAZL gene is associated with spermatogenic failure in the Han Taiwanese population (Teng et al., 2002), providing direct evidence for the role of the DAZL gene in human spermatogenesis. This reference also identified a polymorphism (A/G transition, T12A, Thr₁₂ Ala in exon 2) of the DAZL gene which was not found to be associated with spermatogenic failure in the population studied. A more recent article by the same group reports allele/genotype frequency, LD characteristics, and haplotypes of the DAZL gene occurring in the Han Taiwanese (Teng et al., 2006). The reference reports the association of certain autosomal gene haplotypes with human spermatogenic failure.

In contrast, Tschanter P. et al., 2004 reports that DAZL SNP 386 (T54A) is completely absent in a selected Causacian population. Bartoloni et al. 2004 similarly reports that the T54A polymorphism is absent in an Italian infertile male population. Becherini L. et al. 2004 reports a study of mutations of the entire coding sequence of the DAZL gene in infertile and normospermic men of Italian origin lacking the DAZ gene cluster, to evaluate if DAZL polymorphisms influence the AZFc deletion phenotype. No new mutations were found and the lack of functional role for T12A was confirmed as was the absence of the T54A polymorphism in the population studied. Additionally, Thangaraj et al. 2006 has reported a study of the DAZL gene in an Indian (subcontinent) population. The reference reports that A to G transitions at the previously reported site 260 (T12A), and at a novel site A437G (I71V) in DAZL were not associated with male infertility in the population studied. The previously reported T54A transition was also not found in the population studied.

Van Golde R. J. T. et al 2001 (also published as part of the doctoral Thesis of R. J. T. Van Golde, Katholiehe University Nijmegen N L, January 2002, see Chapter 4) reports a study of the role of DAZLA (Deleted in Azoospermia Like Autosome) in human male subfertility. No mutations were found in the subfertile population studies. Two polymorphisms were identified in intron 4 and 5, which were described as neutral with respect to subfertility. The authors state “(a)ₜ this moment it does not seem relevant to search for possible mutation in the DAZLA gene in clinical practice.”

Tung et al 2006a identified SNP variants in DAZL which are correlated with reproductive parameters in both men and women. Populations studied included women with idiopathic spontaneous premature ovarian failure (premature ovarian failure group), a case control group that contained women with reported ages of ovarian failure/menopause between 28 to 54 years (ovarian failure/menopause group) and a group of infertile men with few or no sperm (oligozoospermia, <20 million sperm/ml), and/or immotile sperm (asthenozoospermia, <50% motile sperm) (infertile male group). Twelve SNPs (single nucleotide polymorphisms) or variants were identified in these populations that were in Hardy-Weinberg equilibrium and had frequencies, of the least common allele, of greater than one percent. Of these SNPs one resulted in a non-synonymous amino acid substitution, six altered nucleotides of the 3′-UTR, and five mapped to introns. A strong association between several SNPs and age at menopause in women and sperm count in men. Tung et al. 2006b reports the identification of four putative missense mutations in human DAZL. These mutations were found only in infertile men and women. Three individuals that were heterozygous for a DAZL mutation reported having children, while two individuals that were homozygous reported no children.

The DAZ gene family, including DAZL, has not as yet been characterized in any farm animal species. It is believed that SNPs and haplotypes comprising SNPs of the DAZL gene have not previously been associated in any organism with any quantitative measure of fertility in males that would be characterized as fertile.

Because of the lack of diagnostic tools or indicators, farm animals with subfertility or infertility are usually not identified until the age when they are expected to breed (Moura and Erickson 2001). And quite often, even if an infertile bull is identified, the bull is usually eliminated from the breeding program immediately without further investigation. There are numerous reports of bull subfertility or infertility in the literature. Most of these studies focused on routine exams of semen quality including sperm counting, structural and morphological analyses. Some of the studies investigated the problem genetically (Donald and Hancock 1953), cytogenetically (Kovacs et al. 1992; Ansari et al. 1993; Villagomez et al. 1993, lannuzzi et al. 2001), or histologically and endocrinologically (Moura and Erickson 2001; Parkinson 2000). Few of the genetic anomalies affecting bull fertility are well characterized. Most anomalies are classified as genetic, based on a preponderance of circumstantial evidence rather than on data from controlled scientific studies (reviewed by Steffen 1997). To date, none of these studies has dealt with the problem at the molecular level. Therefore, there is a significant need to carry out molecular genetic studies of bull subfertility/infertility, especially by a candidate gene (bDAZL) approach.

SNP. A SNP (single nucleotide polymorphism) is a single base substitution of one nucleotide with another, and both versions are observed in the general population at a frequency greater than 1%. (Mutations occur with frequencies less than one percent.) DNA is comprised of only four chemical entities, i.e. A, G, C, T, whose specific chemical order is the alphabet of the genome. An example of a SNP is individual “A” having a sequence GAACCT while individual “B” having a sequence GAGCCT, the polymorphism is an A/G. When chromosomes from two random people are compared, they differ at about one in 1000 DNA sites. Thus when two random haploid genomes are compared, or all the paired chromosomes of one person are compared, there are about three million differences. The most recognized public effort in human was spearheaded by The SNP Consortium (TSC) whose mission was to determine and map about 300,000 evenly spaced single nucleotide polymorphisms within the human genome. Significant efforts to discover SNPs have been made to the farm animal species, especially in cattle and pigs. To date, the bovine SNP database (http://www.animalgenome.org/bioinfo/resources/util/q_bovsnp.html) contains 114,958 SNPs.

Haplotype. A haplotype is the set of SNP alleles along a region of a chromosome. Theoretically there can be many haplotypes in a chromosome region, but recent studies are typically finding only a few common haplotypes. Consider the Scheme 1 below, of a region where six SNPs have been studied; the DNA bases that are the same in all individuals are not shown. The three common haplotypes are shown, along with their frequencies in the population. The first SNP has alleles A and G; the second SNP has alleles C and T. The four possible haplotypes for these two SNPs are AC, AT, GC, and GT. However, only AC and GT are common; these SNPs are said to be highly associated with each other, or they are in a status called Linkage Disequilibrium (LD).

LD describes a situation in which some combinations of alleles or genetic markers occur more or less frequently in a population than would be expected from a random formation of haplotypes from alleles based on their frequencies. Non-random associations between genes at different loci are measured by the degree of linkage disequilibrium.

SCHEME 1

| | |
|---|---|
| ..A..C..A..T..G..T.. | 40% |
| ..A..C..C..G..C..T.. | 30% |
| ..G..T..C..G..G..A.. | 20% |
| several others | 10% |

The cost of genotyping is currently too high for whole-genome association studies that would look at millions of SNPs across the entire genome to see which SNPs are associated with disease. If a region has only a few haplotypes, then only a few SNPs need to be typed to determine which haplotype a chromosome has and whether the region is associated with a disease (http://www.genome.gov/10001665). In our case, all SNPs are located within the DAZL gene that is mapped in the distal region of BTA1q.

SNPs and Diagnostics: Many common diseases in humans are not caused by one genetic variation within a single gene, but are determined by complex interactions among multiple genes, environmental and lifestyle factors. Genetic factors confer susceptibility or resistance to a disease and influence the severity or progression of disease. Since we do not yet know all of the factors involved in these intricate pathways, researchers have found it difficult to develop screening tests for most diseases and disorders, such as diabetes, Alzheimer's disease, arthritis, to name just a few. By studying SNP profiles or haplotypes associated with a disease trait, researchers may begin to reveal relevant genes associated with a disease. Association study can detect and indicate which pattern is most likely associated with the disease-causing genes. Eventually, SNP profiles that are characteristic of a variety of diseases will be established. Then, it will only be a matter of time before physicians can screen individuals for susceptibility to a disease just by analyzing their DNA samples for specific SNP patterns. The SNP effort will serve as the bedrock of pharmacogenomics, the emerging field of personalized medicine: the right drug, in the right dose, to the right person, at the right time (http://las.perkinelmer.com/content/snps/genotyping.asp#snps).

SNP study is also extremely important in organisms other than humans. Within agriculture, genetic modification of the agriculturally important crops (corn, wheat, rice, soybeans, etc.) could lead to improved crop yields at lower cost by reducing the amounts of fertilizer, insecticides, herbicides required. Within microorganisms and viruses, SNPs are known to cause increased drug resistance. Some of the recent E. coli outbreaks are due to new evolving strains of the bacterium. HIV, the causative agent of AIDS, has historically been so difficult to treat with drugs due to very high mutation frequency primarily in the form of SNPs.

In the farm animals, many diseases and most traits, such as semen quality, fertility, milk production, and meat quality, are determined by complex interactions among multiple genes and environmental factors. SNP profiles that are characteristic of a variety of animal traits once identified can be used as DNA molecular markers for marker-assisted selection (MAS).

Marker-Assisted Selection MAS is a method of selecting desirable individuals in a breeding scheme based on DNA molecular marker patterns instead of, or in addition to, their trait values. When used in appropriate situations, it is a tool that can help animal (plant) breeders select more efficiently for desirable animal (crop) traits. MAS has been widely used in plant breeding. In farm animal breeding, although hundreds of thousands of SNPs have been discovered, many of which have not be verified/validated in different populations, very few have been used in the animal breeding system.

Genetic Testing of Cattle. U.S. Pat. No. 7,094,544 relates to molecular markers for identifying potential bovine carriers of Complex Vertebral Malformation (CVM). This patent discusses methods for the detection of genetic markers in genetic material and diagnostic kits for the detection of certain genetic markers.

U.S. Pat. No. 6,673,534 relates to methods for identifying individuals having increased muscle mass or having a predisposition for increased muscle mass by detecting the presence of a variant myostatin nucleotide sequence that is indicative of these traits. The patent discusses methods for the detection of the variant nucleotide sequence and diagnostic kits for the detection of such sequence. Assays and kits are describes as useful in bovine subjects.

U.S. Pat. No. 5,374,523 relates to an assay for determining the presence, in bovine genetic material, of a genetic marker in the bovine somatotropin gene which is indicative of an inheritable trait of increased milk production. The patent discusses methods for detection of the genetic marker and diagnostic kits for use in detecting the genetic marker.

U.S. published application 2006/0211006 relates to the identification of a single nucleotide polymorphism (SNP) with the bovine CAST locus encoding the calpastatin protein which is associated with post-mortem muscle tenderness. The allelic variation of the SNP is a G/C transversion. The patent application describes methods for identifying the genotype of bovine with respect to the CAST locus polymorphic site.

U.S. published application 2005/0123929 relates to methods for genetically detecting improved milk production traits in cattle. The patent application discusses methods for detecting SNP, methods for haplotyping a bovine cell, methods for progeny testing of cattle based on the haplotyping and a method for selective breeding of cattle based on haplotyping of the parent animal. Certain haplotypes are reported to be associated with desired milk production traits.

Fertility Testing in Bulls. U.S. Pat. Nos. 5,962,241, 5,569,581 and U.S. published application 2001/0008764 relate to methods of predicting fertility potential in bulls based on the detection of components in seminal plasma. The patents and application discuss methods used for fertility assessment in bulls and discuss the economic benefits of use of high fertility potential bulls. The '764 application states “(r)egrettably, criteria to accurately select males exhibiting the highest fertility are poorly defined, and the physiological importance of parameters that are reliable indicators of fertility potential is poorly understood. Morphological examination of sperm cells and semen, alone, is insufficient to predict male reproductive success. Taking even single sire situations, and assessing outcomes from artificial insemination, bulls with acceptable semen characteristics by currently accepted evaluations vary widely in their ability to impregnate cows. Variations in fertility between bulls that produce similarly assessed semen contributes greatly to the problem of identifying bulls with the highest fertility potential.”

Sperm viability is one factor used to predict sire fertility. Sperm motility, acrosomal integrity, cervical mucus penetration and cellular content of DNA, enzymes and lipids are employed to assess sperm cell viability (Saacke R G, and J M White). Semen quality tests and their relationship to fertility are discussed in Proc. 4.sup.th Tech Conf Artif Insem Reprod., Natl Assoc Anim Breeders 1972; p 2-7 and Saacke R G. Semen quality in relation to semen preservation. J Dairy Sci 1983; 66:2635-2644). However, these factors are not reliable for prediction of fertility.

Non-return rate is a widely-used, practical measure of relative fertility. Commercial artificial insemination companies, in particular, determine and employ non-return rates to assess their bulls. Non-return rate is a percentage of cows not re-bred to the same bull within 30-60 days post-insemination. Non-return rate is an art-recognized measure of a bull's ability to impregnate cows.

The Breeding Soundness Exam or BSE is also employed to predict fertility. The exam includes assessment of scrotal circumference, sperm motility, sperm morphology and bulls as classified as satisfactory potential breeders, if they meet or exceed certain thresholds (Chenoweth P J, Spitzer J C, and F M Hopkins. A new bull breeding soundness evaluation form. Proc Ann Mtng Soc for Therio, San Antonio, Tex., 1992). The fertility of different bulls which satisfy the BSE requirement may, however, be significantly different.

Assessment of seminal plasma, sperm or use of BSE are applicable to adult animals. The present invention in contrast provides genetic methods for prediction of male fertility that can be used to assess male individual animals at birth (or shortly thereafter) or even in utero.

### SUMMARY OF THE INVENTION

This invention relates to SNPs in the DAZL gene associated with enhanced male fertility in males and haplotypes formed from such SNPs.

Specifically, the invention provides a method for identifying bovine males which will produce semen which will exhibit a higher rate of successfully impregnation by either artificial insemination or natural mating. The method of the invention comprises the steps of:

(a) obtaining a sample of genetic material from an individual male bovine;

(b) assaying in a DAZL gene of that genetic material for the presence of a particular allele of single nucleotide polymorphisms (SNPs) that forms a unique haplotype in the individual male bovine that is associated with decreased fertility thereby identifying that individual male bovine as one that will not exhibit a higher rate of successful impregnation; or

(c) assaying in a DAZL gene of that genetic material for the presence of a particular allele of single nucleotide polymorphisms (SNPs) that forms a unique haplotype in the individual male bovine that is associated with enhanced fertility thereby identifying that individual male bovine as one that will exhibit a higher rate of successful impregnation or both step (b) and (c).

In specific embodiments, the method is used to identify male dairy or beef cattle and the higher rate of successful impregnation is assessed during dairy and beef cattle reproduction.

In specific embodiments, the genotype of the male bovine and its associated haplotype(s) is (are) characterized by the presence of one or more, or preferably two or more, SNPs in the introns, exons, the 5′UTR and 3′UTR regions of the bDAZL gene. In more specific embodiments, the genotype of the male bovine and its associated haplotypes (s) is (are) characterized by the presence of one or more, or preferably two or more, SNPs in the introns, the 5′UTR and 3′UTR regions of the bDAZL gene. In additional more specific embodiments, the genotype of the male bovine and its associated haplotypes is characterized by the presence of one or more, or preferably two or more, SNPs in the introns, and the 3′UTR regions of the bDAZL gene. In other specific embodiments, the genotype of the male bovine and its associated haplotypes are characterized by 2-20 SNP in the regions noted above. In other specific embodiments, the genotype of the male bovine and its associated haplotypes are characterized by three or more, four or more, five or more, six or more, seven or more, eight or more, nine or more, ten or more, eleven or more, twelve or more, thirteen or more, fourteen or more, fifteen or sixteen SNP. In other specific embodiments, bDAZI SNPs are those identified in Table 2A herein.

In specific embodiments, a higher rate of successful impregnation is measured as a higher than average non-return rate or a higher than average pregnancy rate or other art-accepted measurements of male fertility. The term average is the average of the specific population of which the animal under study is a member.

In specific embodiments, haplotypes are those of Tables 6, 7 or 8 herein. In other embodiments, haplotypes are those formed from SNPs of Table 2A herein.

In specific embodiments, the haplotypes are not characterized by an SNP associated with a change in amino acid of the DAZL gene.

The assay methods herein can further comprise a step of genetically identifying potentially infertile bovine males in the population of bovine male animals being assessed by assaying for the presence of a deletion or a change in amino acid in the coding sequence of the DAZL gene. The methods herein can further be combined with known methods for assessment of quality of a bovine male animal for used in breeding and particularly with methods known in the art for assessing bovine male animals as sires for natural service or for use in artificial insemination methods.

In a related embodiment, the invention provides a method for breeding a bovine male animal to obtain male offspring which will produce semen which will exhibit a higher rate of successfully impregnation by either artificial insemination or natural mating which comprises the steps of:

(a) obtaining a sample of genetic material from each of the bovine male and bovine female individuals which are to be bred;

(b) assaying each sample for the presence of a particular allele of single nucleotide polymorphisms (SNPs) that forms a unique haplotype in the individual bovine animals that is associated with decreased male fertility thereby identifying that individual bovine as one that is undesirable for breeding to generate higher fertility male offspring; and/or

(c) assaying for the presence of a particular allele of single nucleotide polymorphisms (SNPs) that forms a unique haplotype in the individual bovine that is associated with enhanced male fertility thereby identifying that individual bovine as one that that is desirable for breeding to generate higher fertility male offspring; and

(d) the results obtained to select pairs of male and female animals for breeding.

In specific embodiments, the haplotype is characterized by three, four, five, six, seven, eight, nine or ten SNPs as described herein. In other embodiments, the haplotype consists essentially of three, four, five, six, seven, eight, nine or ten SNPs as described herein.

In specific embodiments, the invention provides a method for breeding cattle to obtain male offspring which will produce semen which will exhibit a higher rate of successfully impregnation by either artificial insemination or natural mating. The method is particularly useful in breeding of dairy cattle or cattle for beef production.

In specific embodiments, the methods herein can be performed using genetic material obtained from a male bovine (and/or female bovine) at or soon after birth (days or weeks). In specific embodiments, the methods herein can be performed using genetic material obtained from a male bovine (and/or female bovine) in utero.

The invention also related to kits comprising one or more nucleic acid probes or primers useful in identifying the SNPs, haplotypes or genotypes described herein. Kits herein comprise a container and one or more nucleic acid probes or primers as needed. Such probes or primers may be individually packaged within the container for use in one or more assays. The kit can also include instructions, buffers, reagents, tools or other items useful or convenient for carrying out the assay. Those of ordinary skill in the art in view of the information provided herein, in view of sequence information herein and other useful information known in the art can select probes and primers appropriate for use in the methods herein. Those of ordinary skill in the art will further appreciate that any means known in the art for identifying SNPs, haplotypes and/or genotypes can be used in the practice of this invention.

The invention further relates to isolated nucleic acid molecules comprising bDAZL gene sequence of Seq ID NO.1 (FIG. 4) and one or more of the SNP identified in Table 2A herein. In specific embodiments, the isolated nucleic acid molecules comprise at least 15-30 contiguous bases, at least 17 contiguous bases, at least 20 contiguous bases, at least 15 contiguous bases, at least 25 contiguous bases, or at least 30 contiguous bases of the bDAZL gene sequence. In other embodiments, the isolated nucleic acid molecules comprise less than the entire bDAZL sequence. In specific embodiments, the isolated nucleic acid molecules comprise at least 15-30 contiguous bases, but less than the entire bDAZL sequence, at least 17 contiguous bases, but less than the entire bDAZL sequence, at least 20 contiguous bases, but less than the entire bDAZL sequence, at least 15 contiguous bases, but less than the entire bDAZL sequence, at least 25 contiguous bases, but less than the entire bDAZL sequence, or at least 30 contiguous bases of the bDAZL gene sequence, but less than the entire bDAZL sequence. In other specific embodiments, the isolated nucleic acid molecules consist of 15

In additional embodiments, isolated nucleic acid molecules of the invention comprise two or more, three or more, four or more, five or more, six or more, seven or more, eight or more, nine or more, ten or more, eleven or more, twelve or more, thirteen or more, fourteen or more, fifteen or sixteen of the SNP identified in Table 2A. In other embodiments, isolated nucleic acid molecules comprise less than 50, less than 100 or less than 200 contiguous bases of the bDAZL sequence of Seq ID NO.1 (FIG. 4). The invention further provides isolated nucleic acids that are 90% or more, 95% or more or 99% or more identical in sequence to the isolated nucleic acid molecules of bDAZL described herein. In specific embodiments, isolated nucleic acid molecules comprise only a single SNP of Table 2A. In specific embodiments, the contiguous sequence is adjacent the polymorphic site in the nucleic acid molecule. In other embodiments, the polymorphic site is within 4 bases of the center of the nucleic acid molecule. In other embodiments, the polymorphic site is at the center of the nucleic acid molecule. In other embodiments, the polymorphic site is at the 3′-end of the nucleic acid molecule. In specific embodiments, the isolated nucleic acid is between 50-300, 50-100, or 100-200 nucleotides in length. In other embodiments, the isolated nucleic acid molecule is less than 50 nucleotides in length or is greater than 300 nucleotides in length

In other embodiments, the invention provides isolated nucleic acid molecules of sequence complementary to any isolated nucleic acid described hereinabove. These isolated nucleic acids are among other applications useful for the identification of SNPs, haplotypes and genotypes herein.

In specific embodiment, the invention provides isolated nucleic acid primers of Table 1. and Table 13. The invention further provides isolated nucleic acid primers which are 90% or more, 91% or more, 92% or more, 93% or more, 94% or more, 95% or more, 96% or more, 97% or more, 98% or more, or 99% or more identical in sequence to an isolated nucleic acid primer of Table 1 or Table 13.

Additional aspects and embodiments of the invention will become apparent on review of the detailed description and the Examples.

### BRIEF DESCRIPTION OF THE DRAWINGS

FIG. 1 is a graph showing the results of a Oneway (ANOVA) analysis of NRR by genotype.

FIG. 2 is a graph showing the results of a Oneway (ANOVA) analysis of NRR by parental haplotype 1.

FIG. 3 is a graph showing the results of a Oneway (ANOVA) analysis of NRR by parental haplotype 2.

FIG. 4 provides the full-length cDNA (Seq. ID NO.1) and deduced amino acid (Seq. ID NO. 2) sequences of bDAZL. In the nucleotide sequence the upper-case letters represent the coding region, while lower-case letters represent the 5′ and 3′ UTRs. The predicted amino acid sequence is shown in upper case letters beneath the coding sequence. The RNA recognition motif (RRM) (aa 32-117) is underlined. A region that corresponds to the human DAZ repe4 at (aa 167-190) is in italics. The cDNA sequence presented here is the bDAZL transcript variant I (GenBank Acc. No. DQ408764). The region from nt 1,084 to 2,706 (1,623 bp) (lower case letters in italics) in the 3′UTR has been spliced out on the transcript variant 2, resulting in a 1,373, bp mRNA (GenBank Acc. No. DQ408765). The “GUUC” motifs are highlighted in bold/underlined letters.

FIG. 5 illustrates the genomic organization of bovine (A) and human (B) DAZL genes. Genomic organization of bovine (A) and human (B) DAZL genes. (A) The figure was drawn based on the bovine genomic contig BtUn_WGA7606 (NW_{—}001003871.1), the DAZL cDNA and its deduced amino acid sequence. The hatched region in the cDNA 3′ UTR is eliminated in the shorter transcripts (variant 2) by alternative RNA splicing. (B) This was a redrawn figure according to the report from NCBI Map View for the human DAZL gene.

FIG. 6 is a schematic illustration of the conserved segments between BTA1, HSA3 and HSA21, modified from the published cattle-human comparative maps (See Everts-van der Wind et al. 2005 and Itoh et al. 2005). A synteny block of HSA3 from nt 15,457,380 to 18,770,186 that contains the human DAZL gene corresponds to the distal region of BTA1q, supporting the FISH mapping result.

FIG. 7 provides a sequence comparison between bovine DAZL cDNA and its pseudogene. The cDNA and the BTA16 genomic contig (NW_{—}929036) that contains the pseudogene are aligned. Sites that correspond to the insertion or deletion positions on both sequences are indicated. Except for an 874 bp and 2,522 bp insertion and 13 bp deletion in the pseudogene, the two sequences have an overall identity of 87%, indicating that the pseudogene is a cDNA copy of DAZL originated through reverse transcription. Primer pair DAZL602F/1074R indicated on the top of the cDNA amplifies a similar size (473 bp) of PCR products from the cDNA, testis cDNA libraries and bovine genomic DNA.

### DETAILED DESCRIPTION OF THE INVENTION

### Cloning and Characterization of the bDAZL Gene

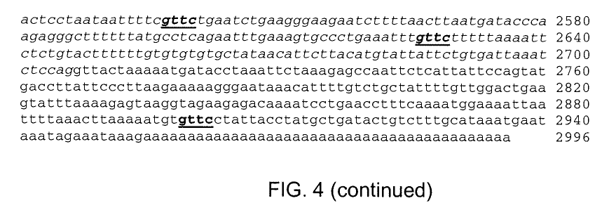

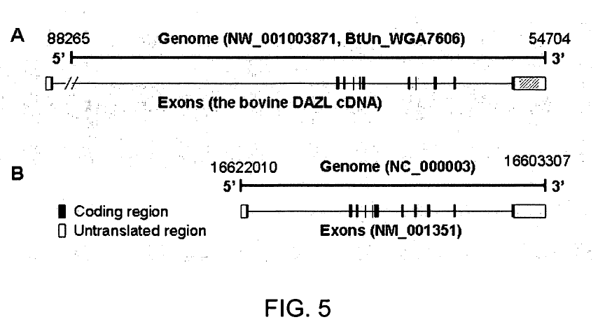

A description of the cloning and sequence analysis of bDAZL is provided in the Examples. The sequences of the full-length cDNA clone and the predicted amino acid sequence of the bDAZL gene is provided in FIG. 4. Additional summary results are provided in FIGS. 5, 6 and 7. The following was determined from this analysis of bDAZL. There is no DAZ gene on the bovine Y chromosome, but there is a DAZL gene on the autosome in the bovine genome. The full-length bDAZL cDNA is predicted to encode a protein of 295 amino acids with an RRM. The deduced protein sequence of bDAZL is 96% similar to human DAZL and 97% similar to mouse Dazl.

Fluorescence in situ hybridization (FISH) maps bDAZL to the distal region on BTA1q. The bDAZL consists of 11 exons and 10 introns, spanning ˜33 kb in the genome. A bDAZL pseudogene was identified on BTA16. Expression analysis of bDAZL in 13 different tissues by RT-PCR shows that two transcripts, variant 1 and 2, of the bDAZL gene are detected only in testis mRNA. The transcript variants are believed to result from alternative RNA splicing, because variant 1 contains an additional 1623 bp insertion in the 3′UTR. A sequence motif “GUUC” that can activate the translation of its own mRNA is present in the 3′UTR in bDAZL variant 2, while four additional “GUUC” motifs are present in variant 1 within the 1623 bp region. These results provided the groundwork for SNP discovery and functional studies of the bDAZL gene in cattle.

bDAZL SNP

Two different approaches were applied in this research. The first approach was an in silico bioinformatics approach. We searched the bovine expressed sequence tags “ESTs” databases available online at TIGR and GenBank using the bDAZL cDNA sequence and identified two ESTs related to bDAZL. These ESTs were compared by sequence alignment. Only one nucleotide substitution (C→G) at nt 2771 of the bDAZL cDNA was identified and was considered as a potential SNP. Because there were not enough bDAZL-related ESTs in the databases, a second approach was applied by pooling DNA samples from fertile bulls as well as some subfertile bulls from one of the world's largest AI companies (The Semex Alliance, Canada) to identify SNPs. The fertile bulls included the top 10 and bottom 10 bulls as assessed by the provided, at least in part by Non-Return Rate, which is a measure of the success of an insemination event. DNA samples pooled from the top 10, bottom 10 bulls, and 4 subfertile bulls were used in PCR (Table 1) and then the PCR fragments were sequenced. In addition, we have collected a DNA sample from an infertile bull, which was also added to the pooled DNA templates (we referred to this as the master pooled DNA).

As both bDAZL transcripts (2996 bp and 1383 bp) are large, spanning ˜33 kb in the genome, the discovery of SNPs was initially focused on all exons and the 3′UTR region that contains the sequence “GUUC” motifs.

TABLE 1

Oligonucleotide primers used
for PCR or SNP genotyping

| | | |
|---|---|---|
| Seq ID No. | Primer name | Sequence |

| | |
|---|---|
| DAZL-IN1F | 5′-GTTCCTCTTCACCTTCTTGG-3′ |
| DAZL-IN2R | 5′-CCTTATGTTTACAACACTACC-3′ |
| DAZL-EX2F | 5′-GGCTATGTTTTACCAGAAGG-3′ |
| DAZL-EX3R | 5′-GACACACCAGTTCGATCC-3′ |
| DAZL-IN3R | 5′-GATTAAGGCAATGTAGGAAC-3′ |
| DAZL-EX4R | 5′-ATCTTCTGCACATCCACGTC-3′ |
| DAZL-IN3F | 5′-CATGTACTGATACGGTGGTG-3′ |
| DAZL-IN4F | 5′-AGTGAAGGTAAGTAAGGTGG-3′ |
| DAZL-EX6R | 5′-TTATCATGGTTGGAGGCTGC-3′ |
| DAZL-EX5R | 5′-ATTGCAGGGCCCAGTTTCAG-3′ |
| DAZL-IN5F | 5′-GGAATTGTTCTTGTTGACAC-3′ |
| DAZL-IN6R | 5′-ACAGCACTAGGATTGTCACG-3′ |
| DAZL-IN6F | 5′-CATGAAAGCACTCACTCAG-3′ |
| DAZL-IN7R | 5′-ACAGTGTCACAGATACGGTC-3′ |
| DAZL-IN7F | 5′-TGACCGTATCTGTGACACTG-3′ |
| DAZL-IN8R | 5′-CAGGCATACTTGATATGGCA-3′ |
| DAZL-IN8F | 5′-GAACCTCAGAACAATACAC-3′ |
| DAZL-IN9R | 5′-GACAGAAGCCATACTAATC-3′ |
| DAZL-IN9F | 5′-ACATACTATGTGACATGGTG-3′ |
| DAZL-IN10R | 5′-CTCACTCTCAGGCTACAGTC-3′ |
| DAZL-IN10F | 5′-GTCTCTTAGGTCATAATGG-3′ |
| DAZL-EX11R1 | 5′-AACTACTCTGACTCTCCTGG-3′ |
| DAZL-EX11R2 | 5′-CTGAATTTCAAGGATCTCAC-3′ |
| DAZL-EX11R3 | 5′-ACTGCTTATACTCCTCCTCC-3′ |
| DAZL-EX11F1 | 5′-AACTGGTTCTGTGTTGAATA-3′ |
| DAZL-EX11F2 | 5′-GAAGGCTGAAACTTTGTCAC-3′ |
| DAZL-EX11F3 | 5′-ATCGTGACAAGCACCTGGAG-3′ |
| DAZL-EX11F4 | 5′-GTCTCATAGGAAGTCACAGG-3′ |
| DAZL-EX11F5 | 5′-TACAAGCACTTCACTTCTCC-3′ |

bDAZL SNPs: By the pooled-DNA-PCR-sequencing approach, we have identified 16 SNPs (Table 2A). Among all exons, only one SNP was identified from the exon 10, nt 953, with a silent T→C substitution. At the protein level, this SNP does not change the serine amino acid. Six SNPs were identified from the 3′UTR region, and five SNPs were identified from the intron sequences adjacent to the exons.

TABLE 2A
Summary of the SNPs detected in the bDAZL gene

| | | | Position in | Genotype |
|---|---|---|---|---|
| SNP # | Substitution | Position | the mRNA | analysis |
| 1 | A→G | Intron 1 | | Yes |
| 2 | G→A | Intron 2 | | No |
| 3 | A→C | Intron 3 | | No |
| 4 | G→A | Intron 3 | | Yes |
| 5 | A→C | Intron 3 | | Yes |
| 6 | A→G | Intron 4 | | No |
| 7 | G→A | Intron 9 | | Yes |
| 8 | G→A | Intron 9 | | Yes |
| 9 | T→C | Exon 10 | 953 | Yes |
| 10 | T→A | 3′ UTR | 1256 | Yes |
| 11 | G→A | 3′ UTR | 1356 | Yes |
| 12 | G→C | 3′ UTR | 1600 | Yes |
| 13 | C→T | 3′ UTR | 1820 | Yes |
| 14 | T→G | 3′ UTR | 2519 | No |
| 15 | C→T | 3′ UTR | 2631 | No |
| 16 | C→G | 3′ UTR | 2771 | No |

Once a SNP is identified, it can be verified by two methods, one is PCR-RFLP method, the other, PCR-sequencing. For the PCR-RFLP, genomic DNA from animals with different genotypes for the SNP is used in PCR. The amplified products are subjected to restriction digestion with an enzyme recognizing the mutation site (SNP). For the PCR-sequencing method, we run PCR using three sub-pooled DNA templates, the top 10, the bottom 10, and the subfertile. The purified PCR fragments were sequenced at Nevada Genomic Center (NGC). The sequences were compared to the original cDNA sequence and the sequence obtained from the master pool to validate the SNPs. Those SNPs that have been confirmed can be used in association studies.

Table 2B provides specific locations of SNPs in bDAZL introns. SNPs were identified in intronic regions adjacent and either upstream or downstream of exons. More specifically SNPs associated with male fertility are found in intronic regions within about 300 bp upstream or downstream of exons of the DAZL gene. 10 SNPs among 25 bulls (top 10, bottom 10, 4 subfertile and 1 infertile) (Table 3) have been genotyed. The genotype results are listed in Table 4.

TABLE 2B
Intronic SNP positions in bDAZL

| SNP | Sub- | | | | |
|---|---|---|---|---|---|
| No. | stitution | Position | Position in the genome | Validation | Genotyped |
| 1 | A/G | Intron 1 | 63 bp upstream of | Yes | Yes |
| | | | exon 2 | | |
| 2 | G/A | Intron 2 | 155 bp downstream of | Pending | No |
| | | | exon 2 | | |
| 3 | A/C | Intron 3 | 226 bp upstream of | Pending | No |
| | | | exon 4 | | |
| 4 | C/T | Intron 3 | 94 bp downstream of | Yes | Yes |
| | | | exon 3 | | |
| 5 | T/G | Intron 3 | 90 bp downstream of | Yes | Yes |
| | | | exon 3 | | |
| 6 | A/G | Intron 4 | 180 bp downstream of | Pending | No |
| | | | exon 4 | | |
| 7 | C/T | Intron 9 | 146 bp downstream of | Yes | Yes |
| | | | exon 9 | | |
| 8 | C/T | Intron 9 | 26 bp downstream of | Yes | Yes |
| | | | exon 9 | | |

TABLE 3
Animals and their fertility performance.

| | | Non-Return | # | | |
|---|---|---|---|---|---|
| | | Rate | daughters | Sire | MGS |
| High NRR | | | | | |
| 200HO1930 | Claynook Bounty | 74 | | Affinity | Aeroline |
| 200HO4649 | Regancrest-LH Mail | 73 | | Durham | Aerostar |
| 200HO3327 | Ladys-Manor Rubix | 73 | | Lee | Rudolph |
| 200HO1229 | Long-Haven Cinder | 73 | | Storm | Highlight |
| 039HO0768 | Comestar Classic | 72 | | Bellwood | Raider |
| 073HO1905 | B-M-Y Frank | 72 | | Leadman | Mark |
| 073HO1918 | Meadow Bridge Million | 72 | | Aerostar | Enhancer |
| 200HO4081 | Richesse Steven | 72 | | Rudolph | Jubilant |
| 200HO3073 | Sunnylodge Allison | 72 | | Aeroline | Grand |
| 073HO2876 | Silky Paxton | 72 | | Mason | Jubilant |
| Low NRR | | | | | |
| 200HO4859 | Jolibois Lance | 57 | | Igniter | Rudolph |
| 073HO2906 | Miley Grammy | 58 | | Duster | Leadman |
| 073HO2712 | Bermath Morgan | 59 | | Astre | Prelude |
| 200HO4278 | Belfast Husky | 59 | | Aeroline | Mason |
| 200HO1521 | Donelea Instinct | 60 | | Lee | Aeroline |
| 200HO4126 | Raypel Havid | 60 | | Midas | Aerostar |
| 200HO4213 | Gen-I-Beq Ciggy | 60 | | Aeroline | Tesk |
| 200HO4338 | Henkeseen Hotmail | 61 | | Rudolph | Holiday |
| 200HO4032 | STBVQ-I Kinshasa | 61 | | Mandel | Leadman |
| 200HO4421 | Terrick Reggie | 62 | | Jolt | Emory |
| Subfertile | | | | | |
| 200HO4058 | Derianne Mediran | 50 | 11 | Celsius | Mascot |
| 200HO5064 | Dudoc Byebye Boss | 54 | 29 | Boss | Rubens |
| 200HO1184 | Walnutlawn Virtuous | 58 | 46 | Rudolph | Lindy |
| 200HO3414 | Gillette Filature | 59 | 46 | Jay | Astre |
| Infertile | | | | | |
| MN No1 | Unknown | 0 | | Unknown | Unknown |

TABLE 4
bDAZL SNP genotyping results

| | | | | | | | | | | |
|---|---|---|---|---|---|---|---|---|---|---|
| | IN1F | IN3R | | IN9R | | IN9F | E11F4 | | E11F5 | |
| | 146 | 241 | 245 | 169 | 285 | 182 | 167 | 265 | 153 | 373 |
| | (G/A) (G/A) | (A/C) | (G/A) | (G/A) | (T/C) | (T/A) | (G/A) | (G/G) | (C/T) | |
| Animal | IN1 | IN3 | IN3′ | IN9 | IN9′ | 953T/C | 1256T/A | 1356G/A | 1600G/C | 1820T/C |

| | | | | | | | | | | | |
|---|---|---|---|---|---|---|---|---|---|---|---|
| Top 10 | 1 | G/A | G | A | G | G | T | A | A | G/G | T |
| | 2 | A | / | / | G | G | C | T | A | C | C |
| | 3 | G/A | G | A | G | G | T | A | G/A | G/G | T |
| | 4 | A | G/A | A/C | G | G | T/C | T/A | A | C | T/C |
| | 5 | A | G | A | G | G/A | T | A | G/A | C | T |
| | 6 | G/A | / | / | G | G/A | T | A | / | G/C | T |
| | 7 | A | G/A | A/C | G | G | T/C | T/A | A | C | T/C |
| | 8 | A | / | / | G/A | G | T | A | A | C | T |
| | 9 | A | G/A | A/C | G | G | T/C | T/A | A | C | T/C |
| | 10 | A | G | A | G/A | G/A | T | A | G/A | C | T |
| Bottom 10 | 1 | A | G | A | G | G | T | A | G/A | C | T |
| | 2 | A | G/A | A/C | G | G/A | T/C | T/A | A | C | T/C |
| | 3 | A | G | A | G/A | G | T | A | A | C | T |
| | 4 | G/A | G | A | G | G/A | T | A | G/A | G/C | T |
| | 5 | G/A | / | / | G | G | T | A | A | G/C | T |
| | 6 | G/A | G | A | G/A | G | T | A | A | G/C | T |
| | 7 | G | G | A | G | G | T | A | A | G | T |
| | 8 | A | / | / | G | G/A | T | A | A | C | T |
| | 9 | G/A | G/A | A/C | G | G | T/C | T/A | A | G/C | T/C |
| | 10 | A | G/A | A/C | G | G | T/C | T/A | A | C | T/C |
| Sub-fertile | 1 | A | G/A | A/C | G/A | G/A | T | A | A | C | T |
| | 2 | A | / | / | G/A | G | T | A | G/A | C | T |
| | 3 | G/A | G | A | G | G | T | A | A | G/G | T |
| | 4 | A | / | / | G | G/A | T/C | T/A | G/A | C | T/C |
| Infertile | 1 | / | G/A | A/C | G | G/A | T/C | T/A | A | G/G | T/C |

TABLE 5
Genotype and haplotype analysis

| | | | | | |
|---|---|---|---|---|---|
| | | | | Probability | |
| Individual | | | | of Gametic | |
| ID | Genotype | Parental Haplotype 1 | Parental Haplotype 2 | Assignment | QFert* |

| | | | | | |
|---|---|---|---|---|---|
| 1 | A-G-A-G-G-T-A-A-C-T/ | A-G-A-G-G-T-A-A-C-T | G-G-A-G-G-T-A-A-G-T | 1 | 74 |
| | G-G-A-G-G-T-A-A-G-T | | | | |
| 2 | A-A-C-G-G-C-T-A-C-C/ | A-A-C-G-G-C-T-A-C-C | A-A-C-G-G-C-T-A-C-C | 1 | 73 |
| | A-A-C-G-G-C-T-A-C-C | | | | |
| 3 | A-G-A-G-G-T-A-G-C-T/ | A-G-A-G-G-T-A-G-C-T | G-G-A-G-G-T-A-A-G-T | 1 | 73 |
| | G-G-A-G-G-T-A-A-G-T | | | | |
| 4 | A-A-C-G-G-C-T-A-C-C/ | A-A-C-G-G-C-T-A-C-C | A-G-A-G-G-T-A-A-C-T | 1 | 73 |
| | A-G-A-G-G-T-A-A-C-T | | | | |
| 5 | A-G-A-G-A-T-A-G-C-T/ | A-G-A-G-A-T-A-G-C-T | A-G-A-G-G-T-A-A-C-T | 0.997352001 | 72 |
| | A-G-A-G-G-T-A-A-C-T | | | | |
| 6 | A-G-A-G-A-T-A-G-C-T/ | A-G-A-G-A-T-A-G-C-T | G-G-A-G-G-T-A-A-G-T | 0.661063159 | 72 |
| | G-G-A-G-G-T-A-A-G-T | | | | |
| 7 | A-A-C-G-G-C-T-A-C-C/ | A-A-C-G-G-C-T-A-C-C | A-G-A-G-G-T-A-A-C-T | 1 | 72 |
| | A-G-A-G-G-T-A-A-C-T | | | | |
| 8 | A-G-A-A-G-T-A-A-C-T/ | A-G-A-A-G-T-A-A-C-T | A-G-A-G-G-T-A-A-C-T | 1 | 72 |
| | A-G-A-G-G-T-A-A-C-T | | | | |
| 9 | A-A-C-G-G-C-T-A-C-C/ | A-A-C-G-G-C-T-A-C-C | A-G-A-G-G-T-A-A-C-T | 1 | 72 |
| | A-G-A-G-G-T-A-A-C-T | | | | |
| 10 | A-G-A-A-G-T-A-A-C-T/ | A-G-A-A-G-T-A-A-C-T | A-G-A-G-A-T-A-G-C-T | 1 | 72 |
| | A-G-A-G-A-T-A-G-C-T | | | | |
| 11 | A-G-A-G-G-T-A-A-C-T/ | A-G-A-G-G-T-A-A-C-T | A-G-A-G-G-T-A-G-C-T | 1 | 57 |
| | A-G-A-G-G-T-A-G-C-T | | | | |
| 12 | A-A-C-G-A-C-T-A-C-C/ | A-A-C-G-A-C-T-A-C-C | A-G-A-G-G-T-A-A-C-T | 0.986378256 | 58 |
| | A-G-A-G-G-T-A-A-C-T | | | | |
| 13 | A-G-A-A-G-T-A-A-C-T/ | A-G-A-A-G-T-A-A-C-T | A-G-A-G-G-T-A-A-C-T | 1 | 59 |
| | A-G-A-G-G-T-A-A-C-T | | | | |
| 14 | A-G-A-G-A-T-A-G-C-T/ | A-G-A-G-A-T-A-G-C-T | G-G-A-G-G-T-A-A-G-T | 1 | 59 |
| | G-G-A-G-G-T-A-A-G-T | | | | |
| 15 | A-G-A-G-G-T-A-A-C-T/ | A-G-A-G-G-T-A-A-C-T | G-G-A-G-G-T-A-A-G-T | 1 | 60 |
| | G-G-A-G-G-T-A-A-G-T | | | | |
| 16 | A-G-A-A-G-T-A-A-C-T/ | A-G-A-A-G-T-A-A-C-T | G-G-A-G-G-T-A-A-G-T | 1 | 60 |
| | G-G-A-G-G-T-A-A-G-T | | | | |
| 17 | G-G-A-G-G-T-A-A-G-T/ | G-G-A-G-G-T-A-A-G-T | G-G-A-G-G-T-A-A-G-T | 1 | 60 |
| | G-G-A-G-G-T-A-A-G-T | | | | |
| 18 | A-A-C-G-A-T-A-A-C-T/ | A-A-C-G-A-T-A-A-C-T | A-G-A-G-G-T-A-A-C-T | 0.978669963 | 61 |
| | A-G-A-G-G-T-A-A-C-T | | | | |
| 19 | A-A-C-G-G-C-T-A-C-C/ | A-A-C-G-G-C-T-A-C-C | G-G-A-G-G-T-A-A-G-T | 1 | 61 |
| | G-G-A-G-G-T-A-A-G-T | | | | |
| 20 | A-A-C-G-G-C-T-A-C-C/ | A-A-C-G-G-C-T-A-C-C | A-G-A-G-G-T-A-A-C-T | 1 | 62 |
| | A-G-A-G-G-T-A-A-C-T | | | | |
| 21 | A-A-C-G-A-T-A-A-C-T/ | A-A-C-G-A-T-A-A-C-T | A-G-A-A-G-T-A-A-C-T | 0.953114981 | 50 |
| | A-G-A-A-G-T-A-A-C-T | | | | |
| 22 | A-G-A-A-G-T-A-A-C-T/ | A-G-A-A-G-T-A-A-C-T | A-G-A-G-G-T-A-G-C-T | 0.999999911 | 54 |
| | A-G-A-G-G-T-A-G-C-T | | | | |
| 23 | A-G-A-G-G-T-A-A-C-T/ | A-G-A-G-G-T-A-A-C-T | G-G-A-G-G-T-A-A-G-T | 1 | 58 |
| | G-G-A-G-G-T-A-A-G-T | | | | |
| 24 | A-A-C-G-G-C-T-A-C-C/ | A-A-C-G-G-C-T-A-C-C | A-G-A-G-A-T-A-G-C-T | 0.838745952 | 59 |
| | A-G-A-G-A-T-A-G-C-T | | | | |
| 25 | A-A-C-G-A-C-T-A-C-C/ | A-A-C-G-A-C-T-A-C-C | G-G-A-G-G-T-A-A-G-T | 1 | 0 |
| | G-G-A-G-G-T-A-A-G-T | | | | |

*QFert stands for quantitative measurement of fertility; the values listed here are the non-return rate (NRR).

Association Study of bDAZL SNPs with Male Fertility.

Genotype and haplotype analysis. The SAS/JMP software was used to analyze the genotype, haplotype and trait association of the 25 animals studied. Table 5 shows the genotype and the most probable gametic assignments, along with NRR (non-return rate).

Binary haplotype association analysis—-High NRR vs Subfertile (sf) group. The linkage disequilibrium (LD) test for allelic association between the high NRR group and subfertile group indicates that there is a very significant LD among the 10 SNPs and their alleles (p<0.0001) (Table 6). The test for marker-trait association also reveals a significant link between the SNPs and the fertility trait (p<0.005). Further tests for haplotype-trait association have identified one haplotype (A-A-C-A-A-T-A-A-C-T) that is strongly associated to subfertile animals (Table 6) (p<0.001).

TABLE 6
Test for allelic association and marker-trait association between
high and sf.
Test for Allelic Associations

| | | | | Pr > |
|---|---|---|---|---|
| Hypothesis | DF | LogLike | Chi-Square | ChiSq |
| HO: No Association | 10 | −99.66402 | | |
| H1: Allelic Associations | 53 | −39.87615 | 119.5757 | <.0001 |

Test for Marker-Trait Association

| Trait | Trait | Num | | | Chi- | Pr > |
|---|---|---|---|---|---|---|
| Number | Value | Obs | DF | LogLike | Square | ChiSq |
| *1 | high | 10 | 44 | −25.36266 | | |
| *2 | sf | 4 | 18 | −2.77259 | | |
| | Combined | 14 | 53 | −39.87615 | 23.4818 | 0.0052 |

*High NRR/Subfertile

TABLE 7

Tests for Haplotype-Trait Association
between high NRR and subfertile groups.

| | |
|---|---|
| | Chi- |
| Frequencies | Square Pr> |

| | | | | | | |
|---|---|---|---|---|---|---|
| No. | Haplotype | Seq.ID No | High NRR | Subfertile | Combined | ChiSq |

| | | | | | | |
|---|---|---|---|---|---|---|
| 1 | A-A-C-A-A-T-A-A-C-T | 0 | 0.25 | 0.03571 | 11.4074 | 0.0007 |
| 2 | A-A-C-G-G-C-T-A-C-C | 0.24999 | 0 | 0.21428 | 2.3333 | 0.1266 |
| 3 | A-G-A-A-A-T-A-G-C-T | 0.02357 | 0 | 0.01681 | 0.1921 | 0.6612 |
| 4 | A-G-A-A-G-T-A-A-C-T | 0.07643 | 0 | 0.05461 | 0.6465 | 0.4214 |
| 5 | A-G-A-G-A-T-A-G-C-T | 0.12643 | 0 | 0.12605 | 1.1538 | 0.2827 |
| 6 | A-G-A-G-G-T-A-A-C-T | 0.37357 | 0.5 | 0.37395 | 0.5429 | 0.4612 |
| 7 | G-G-A-G-G-T-A-A-G-T | 0.075 | 0.25 | 0.09524 | 2.3187 | 0.1278 |
| 8 | G-G-A-G-G-T-A-G-G-T | 0.075 | 0 | 0.04762 | 0.7306 | 0.3927 |

Binary haplotype association analysis—Low NRR vs Subfertile group. Similar analysis indicates that there is no significant allelic association between low NRR and subfertile groups. This seems to be reasonable as the NRR overlaps between the low NRR (range from 57 to 62) and the subfertile group (range from 50 to 59) (Table 3). However, the test for marker-trait association (Table 8) still shows a significant link between the SNPs and the fertility trait (p=0.0124). Once again, the haplotype (A-A-C-A-A-T-A-A-C-T) is strongly associated to subfertile animals (Table 10) (p<0.0001). The comparison between Low NRR and Subfertile also identified two haplotypes that are associated to the low NRR (p<0.0001) (Table 9).

TABLE 8
Test for allelic association and marker-trait association
between low and sf.
Test for Allelic Associations

| | | | | Pr > |
|---|---|---|---|---|
| Hypothesis | DF | LogLike | Chi-Square | ChiSq |
| HO: No Association | 10 | −95.92115 | | |
| H1: Allelic | 181 | −43.62811 | 104.5861 | 1.0000 |
| Associations | | | | |

Test for Marker-Trait Association

| Trait | Trait | Num | | | Chi- | Pr > |
|---|---|---|---|---|---|---|
| Number | Value | Obs | DF | LogLike | Square | ChiSq |
| 1 | low | 10 | 174 | −28.81227 | | |
| 2 | sf | 4 | 18 | −2.77259 | | |
| | Combined | 14 | 181 | −43.62811 | 24.0865 | 0.0124 |

Binary haplotype association analysis—High NRR vs Low. Although there are no significant allelic association and marker-trait association between the high and low NRR groups (Table 10), we still identified two haplotypes that are significantly associated to the high fertility (p<0.0001), and two haplotypes that are significantly associated to the low fertility (p<0.0001) (Table 11).

TABLE 9

Tests for Haplotype-Trait Association
between low NRR and subfertile groups.

| | |
|---|---|
| Frequencies | Chi-Square |

| | | | | | | | |
|---|---|---|---|---|---|---|---|
| No. | Haplotype | Seq ID No. | Low NRR | Subfertile | Combined | Pr> | ChiSq |

| | | | | | | |
|---|---|---|---|---|---|---|
| 1 | A-A-C-A-A-T-A-A-C-T | 0 | 0.25 | 0.00068 | 731.7467 | <.0001 |
| 2 | A-A-C-G-A-C-T-A-C-C | 0.01094 | 0 | 0.05356 | 1.1694 | 0.2795 |
| 3 | A-A-C-G-G-C-T-A-C-C | 0.13906 | 0 | 0.0893 | 1.3935 | 0.2378 |
| 4 | A-G-A-A-G-T-A-A-C-T | 0.1 | 0 | 0.14218 | 1.6176 | 0.2034 |
| 5 | A-G-A-G-A-T-A-A-C-T | 0.08906 | 0 | 0.00002 | 10445 | <.0001 |
| 6 | A-G-A-G-A-T-A-G-C-T | 0.00021 | 0 | 0.05358 | 1.5764 | 0.2093 |
| 7 | A-G-A-G-G-T-A-A-C-T | 0.31073 | 0.5 | 0.25068 | 3.0316 | 0.0817 |
| 8 | A-G-A-G-G-T-A-G-C-T | 0.05 | 0 | 0.08928 | 1.1638 | 0.2807 |
| 9 | G-G-A-G-A-T-A-G-G-T | 0.04979 | 0 | 0 | 6858979 | <.0001 |
| 10 | G-G-A-G-G-T-A-A-G-T | 0.25021 | 0.25 | 0.25 | 4.57E-06 | 0.9983 |
| | | 1 | 1 | 1 | | |

TABLE 11

Tests for Haplotype-Trait Association
between high and low NRR groups.

| | |
|---|---|
| Frequencies | Pr> |

| | | | | | | |
|---|---|---|---|---|---|---|
| Seq ID No. | Haplotype | High NRR | Low NRR | Combined | Chi-Square | ChiSq |

| | | | | | |
|---|---|---|---|---|---|
| A-A-C-G-A-C-T-A-C-C | 0 | 0.01094 | 0 | 20339 | <.0001 |
| A-A-C-G-G-C-T-A-C-C | 0.24999 | 0.13906 | 0.2 | 0.7766 | 0.3782 |
| A-G-A-A-A-T-A-G-C-T | 0.02382 | 0 | 0.00015 | 73.5609 | <.0001 |
| A-G-A-A-G-T-A-A-C-T | 0.07618 | 0.1 | 0.09985 | 0.1246 | 0.7241 |
| A-G-A-G-A-T-A-A-C-T | 0 | 0.08906 | 0.06382 | 1.5766 | 0.2092 |
| A-G-A-G-A-T-A-G-C-T | 0.12618 | 0.00021 | 0.08602 | 2.2836 | 0.1307 |
| A-G-A-G-G-T-A-A-C-T | 0.37382 | 0.31073 | 0.27204 | 1.1974 | 0.2739 |
| A-G-A-G-G-T-A-G-C-T | 0 | 0.05 | 0.05311 | 1.1257 | 0.2887 |
| G-G-A-G-A-T-A-G-G-T | 0 | 0.04979 | 0 | 36273 | <.0001 |
| G-G-A-G-G-T-A-A-G-T | 0.075 | 0.25021 | 0.22493 | 2.6521 | 0.1034 |
| G-G-A-G-G-T-A-G-G-T | 0.075 | 0 | 0.00007 | 1699 | <.0001 |
| | 1 | 1 | 1 | | |

TABLE 10
Test for allelic association and marker-trait association between
high and low.
Test for Allelic Associations

| | | | | Pr > |
|---|---|---|---|---|
| Hypothesis | DF | LogLike | Chi-Square | ChiSq |
| HO: No Association | 10 | −137.36376 | | |
| H1: Allelic Associations | 177 | −57.85211 | 159.0233 | 0.6580 |

Test for Marker-Trait Association

| Trait | Trait | Num | | | Chi- | Pr > |
|---|---|---|---|---|---|---|
| Number | Value | Obs | DF | LogLike | Square | ChiSq |
| 1 | high | 10 | 44 | −25.36258 | | |
| 2 | low | 10 | 174 | −28.81227 | | |
| | Combined | 20 | 177 | −57.85211 | 7.3545 | 1.0000 |

Binary haplotype association analysis—High NRR vs infertile The LD test for allelic association between the high NRR group and infertile group, once again, indicated that there is a very significant LD among the 10 SNPs and their alleles (p<0.0001) (Table 12). Because only one infertile animal was used in the association study, the statistical tests for marker-trait and haplotype-trait associations could not be completed. However, this is not inconsistent with the fact that the haplotype is associated with the fertility trait as the infertile animal posses some unique haplotypes.

TABLE 12
Test for allelic association and marker-trait association between
high and infertile.
Test for Allelic Associations

| | | | | Pr > |
|---|---|---|---|---|
| Hypothesis | DF | LogLike | Chi-Square | ChiSq |
| HO: No Association | 10 | −71.62354 | | |
| H1: Allelic Associations | 44 | −27.26880 | 88.7095 | <.0001 |

Test for Marker-Trait Association

| Trait | Trait | Num | | | | Pr > |
|---|---|---|---|---|---|---|
| Number | Value | Obs | DF | LogLike | Chi-square | ChiSq |
| 1 | high | 10 | 44 | −25.36260 | | |
| 2 | sf | 1 | — | 0 | | |
| | Combined | 11 | 44 | −27.26880 | | |

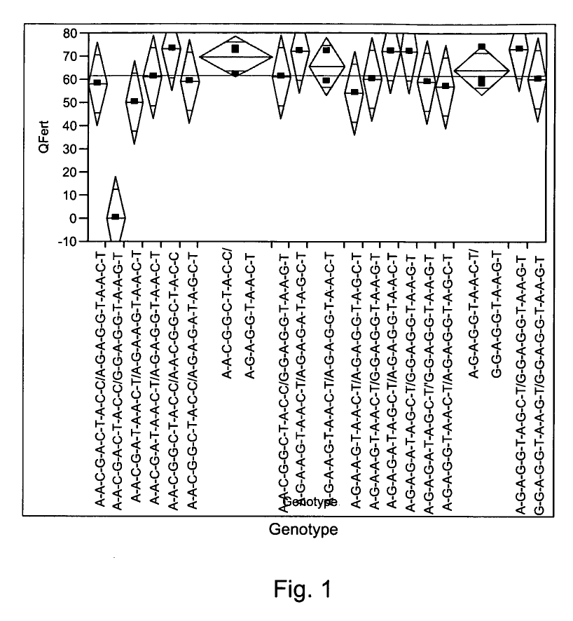

“Oneway ANOVA” analysis of association between genotypes and the fertility trait. Based on information in Table 5, we have carried out a “One-way ANOVA” analysis to study the relationship between the genotypes and the quantitative measurement of NRR. FIG. 1 shows the Oneway ANOVA analysis results with a significant statistical support (p=0.0252), indicating that the bDAZL gene is strongly correlated to the fertility trait.

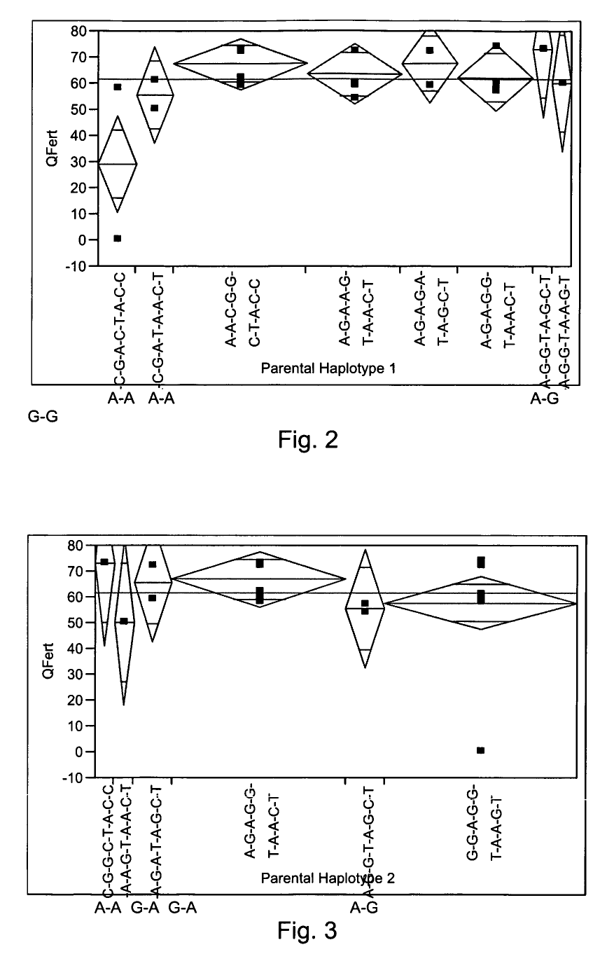

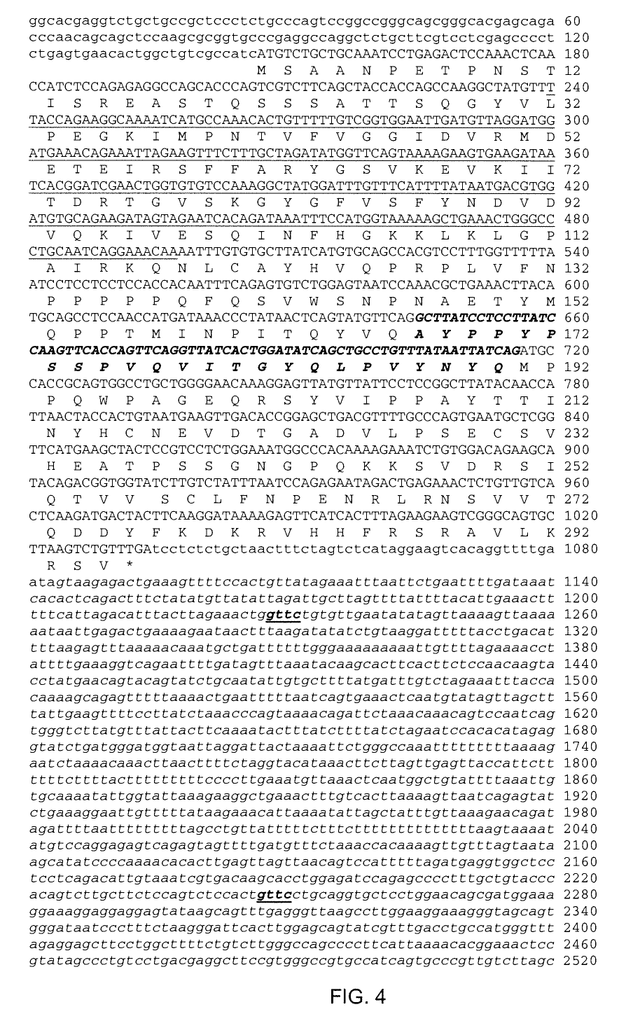

“Oneway ANOVA” analysis of association between parental haplotype 1 and the fertility trait. Similarly, we have carried out a “Oneway ANOVA” analysis to study the relationship between the parental haplotypes and the quantitative measurement of NRR (FIGS. 2 and 3). We found that the parental haplotype 1 is significantly correlated to the NRR measurements (FIG. 2), but not the parental haplotype 2 (FIG. 3).

Summary of the haplotype-trait association study. The LD test for allelic association indicated that there is a very significant LD among the 10 SNPs and their alleles. The marker-trait association test indicated that the SNPs from the bDAZL gene are significantly associated to the fertility trait. The test for haplotype-trait association indicated that different haplotypes are very significantly associated to the fertility trait.

We found that one haplotype (A-A-C-A-A-T-A-A-C-T) (Seq ID No. 3) is unique to the subfertile animals, and several haplotypes either linked to the high, low NRR, subfertile, or infertile.

The bDAZL SNPs and their haplotypes can be used in MAS for sire early selection for the male fertility trait.

Diagnostic kits for male fertility selection using the bDAZL SNPs. Once SNPs from the bDAZL gene have been, identified, validated and trait-association analyzed, a set of the most important SNPs, as described herein, that have a major impact on the genetic variation of the fertility trait can be selected. PCR primers to genotype the SNPs will be collected together. Methods for selecting nucleic acids useful as probes and primers for detection of SNPs and haplotypes are known in the art. For example, U.S. patent applications 2005/0123929 and 2006/0211006 describe such methods. These patent applications are incorporated by reference herein in their entirety to describe such methods.

Extension of the DAZL gene research to other major farm animals. The fact that orthologous genes (boule, Xdazl, DAZ and DAZL) in fly, frog, mouse and human have a similar function, and the success of the human DAZ and DAZL transgenes to partially rescue the mouse Dazl null phenotype indicate that the structures and functions of the DAZ/DAZL proteins are highly conserved among species. This fact raised the possibility that DAZL may also play a fundamental role in spermatogenesis in bovine and other mammalian species as well. The research findings described herein not only confirm that the bovine DAZL gene does play an essential role in male fertility in bovine, indicating a similar role in other mammals, but also have demonstrated that SNPs and combinations of SNPs in the DAZL gene are associated with decreased or enhanced fertility in animals normally considered to be fertile. These results indicate that analogous SNPs in the DAZL genes in pig, sheep, horse, goats and other farm animals as well as in other domesticated animals that are subjected to breeding programs, such as dogs, exhibit similar association and can be used for identifying males with enhanced fertility.

Based on the results in hand, DAZL gene sequences and mRNA in various animals, particularly mammals, e.g., pig, sheep, horse, goat, and dog, will be conserved in structure and highly conserved in sequence to the human, mouse and bovine DAZL. While the specific SNPs in a given gene or genome region are not typically conserved among species, SNPs in the DAZL regions identified herein, particularly in the introns and regulatory sequences of the gene of other animals, will exhibit similar function to those identified in bDAZL herein for the identification of males with enhanced fertility as discussed herein. Based on the information provided herein and the availability of DAZL gene sequences of a given animal species, SNPs exhibiting the function of the SNPs herein can now be readily identified and used as described herein to identify haplotypes and genotypes associated with enhanced male fertility. Such SNPs, haplotypes and genotypes can be used as described herein to identify males of various non-human animals, particularly those of non-human mammals, and more particularly those of horses, pigs, sheep, goats and dogs.

The reproductive process is central to beef productivity. One of the most important components in the animal reproductive process is male fertility. However, male (such as bull) fertility has not been studied at the molecular genetic level. Our study aimed to address the male infertility/subfertility problem in beef cattle with emphasis on a candidate gene bDAZL. The results have provided significant implications and indications for identifying enhanced male fertility in bovine and other non-human animals.

Functional studies of the DAZ/DAZL gene in fly, frog, mouse and human all support a hypothesis that the bDAZL gene plays an important role in male spermatogenesis and fertility in cattle and other farm animal species. Results from this study have tested this hypothesis and provided direct evidence in the function of this gene in bull spermatogenesis and fertility. The results of this study extend, however, to demonstrating that sequence variations in this gene are associated with quantitatively measurable differences in fertility of males otherwise considered to be fertile.

The identification of SNPs from bDAZL and the confirmation of the SNP haplotype-fertility trait association have lead to the design of marker assisted selection (MAS) to select sires at an early age in breeding. This, in turn, will significantly reduce the breeding cost, as males with subfertility or infertility are usually not identified until the age when they are expected to breed.

In human, SNP haplotype-diseases association study is becoming more common. The diagnostic purpose is to genetically screen the patient to prevent or diagnose the diseases early. In short, it is a case-by-case study in humans. In animal breeding, the main goal is to improve the entire population or the breeding value of a population by selecting animals with a favorable genotype and eliminating animals with an unfavorable genotype. The top 10 and bottom 10 bulls from Semex Alliance are not only normal bulls, but the best bulls in Canada (perhaps in the world). The haplotypes we found in this study not only allow us to identify animals that are infertile or subfertile, but also allow us to select animals that have a high NRR or low NRR. The latter is even more important for ranchers and animal breeders to select animals with the best performance.

The designation and application of a diagnostic kit for male fertility selection is not limited to cattle. Additional studies in other farm animal species as described above, based on information provided herein, will yield further species-specific diagnostic kits.

A portion of the work described herein has been published as Liu, W.-S., Wang, A., Uno, Y., Galtz, D., Beattie, C. W., Ponce de León, F. A. (2007) Genomic structure and transcript variants of the bovine DAZL gene. Cytogenetic and Genome Research 116 (1-2), 65-71 (see The Examples below). This reference is specifically incorporated by reference herein in its entirety to provide additional details of the cloning and analysis of bDAZL.

It is recognized by those skilled in the art that DNA sequences may vary due to the degeneracy of the genetic code and codon usage. Additionally, it will be recognized by those skilled in the art that allelic variations may generally occur in the DNA sequences which will not significantly change activity of the amino acid sequences of the peptides which the DNA sequences encode or affect phenotype of the organism. (Noting however that this invention relates to certain specific SNPs associated with significant changes in phenotype). All such equivalent DNA sequences are included within the scope of this invention. It will be understood ti the context of this invention and the nucleic acid molecules that for applications herein that the sequence of certain regions of the nucleic acid molecules of this invention is associated with the function and use of the nucleic acid molecules. Variations in sequence in such regions which are functional in applications herein are not preferred in this invention and in general not result in functionally equivalent nucleic acid molecules.

Hybridization procedures are useful for identifying polynucleotides with sufficient homology to the subject sequences to be useful as taught herein. The particular hybridization techniques is not essential to the subject invention. As improvements are made in hybridization techniques, they can be readily applied by one of ordinary skill in the art.

Some of the nucleic acid molecules of this invention are useful as probes and as illustrated specifically herein as primers for PCT techniques. A probe and sample are combined in a hybridization buffer solution and held at an appropriate temperature until annealing occurs. Thereafter, the membrane is washed free of extraneous materials, leaving the sample and bound probe molecules typically detected and quantified by autoradiography and/or liquid scintillation counting. As is well known in the art, if the probe molecule and nucleic acid sample hybridize by forming a strong non-covalent bond between the two molecules, it can be reasonably assumed that the probe and sample are essentially identical, or completely complementary if the annealing and washing steps are carried out under conditions of high stringency. The probes detectable label provides a means for determining whether hybridization has occurred. Alternatively, hybridization may be detected by virtue of priming in a polymerase chain assay. One of ordinary skill in the art understands in general how to employ nucleic acid molecules as probes pr primers without resort to undo experimentation.

Various degrees of stringency of hybridization can be employed. The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well know in the art, as described, for example in Keller, G. H., M. M. Manak (1987) DNA Probes, Stockton Press, New York, N.Y., pp. 169-170, hereby incorporated by reference.

Polymerase Chain Reaction (PCR) is a repetitive, enzymatic, primed synthesis of a nucleic acid sequence. This procedure is well known and commonly used by those skilled in this art [see, e.g., Mullis, U.S. Pat. Nos. 4,683,195, 4,683,202, and 4,800,159; Saiki et al. (1985) Science 230:1350-1354]. PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

As used herein percent sequence identity of two nucleic acids is determined using the algorithm of Altschul et al. (1997) Nucl. Acids Res. 25: 3389-3402; see also Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See the National Center for Biotechnology Information on the internet.

When a Markush group or other grouping is used herein, all individual members of the group and all combinations and subcombinations possible of the group are intended to be individually included in the disclosure. A number of specific groups are described herein. It is intended that all combinations and subcombinations of the specific groups of defined are individually included in this disclosure. All known variants of nucleic acids are encompassed herein. The invention includes isotopic variants, including radioisotopes of molecules described herein. Molecules described herein can be radiolabelled or otherwise labeled or tagged as known in the art for use in assays herein. Methods for making such isotopic, radiolabelled and tagged variants are known in the art. For example, tagging technology such as TaqMan® systems (Applied Biosystems), and SYBR® Green (Invitrogen) are frequently used in SNP analysis.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. When the broad term “comprising” is used herein, it is intended to encompass the narrower terms “consisting essentially of” and “consisting of.” Thus, the phrase “comprising A and B” encompasses the narrower phrase “consisting essentially of A and B” and the even narrower phrase “consisting of A and B.” The use of the term comprising herein is intended to also provide support for “consisting essentially of” and “consisting of.” The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All references cited herein are hereby incorporated by reference to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention.

### THE EXAMPLES

The Deleted in AZoospermia-Like gene (DAZL) is a member of the Deleted in AZoospermia (DAZ) family, which consists of three genes: BOLL, DAZ and DAZL. It is believed that the ancestor member of the family is BOLL, which gave rise to DAZL via duplication prior to the divergence of vertebrates and invertebrates (Cauffman et al., 2005). Later, during primate evolution, the autosomal DAZL gene gave rise to DAZ on the Y chromosome by transposition, repeat amplification and pruning (Saxena et al., 1996; Shan et al., 1996; Gromoll et al., 1999). As a result, DAZ has been identified only in Old World monkeys and great apes, while DAZL is present in all vertebrates (Cooke et al., 1996; Saxena et al., 1996).

The DAZ family is expressed exclusively in the germ cells encoding proteins that contain a highly conserved RNA-recognition motif (RRM) and a unique DAZ repeat of 24 amino acid (aa) residues (reviewed by Yen, 2004). DAZ genes are expressed only in the testis (Reijo et al., 1995), while DAZL is expressed in both the testis and the ovary (Seligman and Page, 1998; Dorfman et al., 1999; Cauffman et al., 2005). Both DAZ and DAZL are detected in the nuclei of primordial germ cells (PGCs) in fetal gonads (Xu et al., 2001), and are believed to function in the development of PGCs and in germ cell differentiation and maturation (reviewed by Yen, 2004). Mutations in these genes have been linked to infertility in several species. In flies, loss of function of the boule gene, an ortholog of DAZ/DAZL, leads to male sterility (Eberhart et al., 1996). In frogs, inhibition of Xdazl (Xenopus daz-like) leads to defective migration and a reduction in PGCs (Houston and King, 2000). In mice, a disruption of Dazl leads to prenatal loss of all germ cells in both sexes during prenatal germ cell development, and hence infertility (Ruggiu et al., 1997). Further, the infertile phenotype of Dazl null mouse (Dazl—/—) can be partially rescued by a human DAZ transgene (Slee et al., 1999). As the gene name suggests, deletion or microdeletion in DAZ gene(s) on the Y chromosome leads to severe spermatogenic failure and infertility in men (Reijo et al., 1995; Ferlin et al., 1999), and DAZL transcripts in the testes are lower in men with spermatogenic failure compared with fertile men (Lin et al., 2001). A polymorphism (A/G transition) occurring in the RNA binding domain of DAZL confers susceptibility to severe spermatogenic failure in humans, providing direct evidence for the role of the DAZL gene in human spermatogenesis (Teng et al., 2002). However, the DAZL gene has not been characterized in any farm animal species.

To examine the role of the DAZL gene in fertility/infertility and to facilitate the isolation of potential single-nucleotide polymorphisms (SNPs) from DAZL for bull fertility selection, we have cloned and characterized the bovine DAZL (bDAZL) gene. We have found that the bDAZL is highly conserved with the human and mouse sequence, maps to the distal region of the bovine chromosome (BTA) 1, and is expressed only in the testis in cattle.

Materials and Methods. Development of a DAZ gene probe and isolation of bovine BAC clones containing DAZL. We designed a pair of PCR primers from human DAZ2 mRNA (GenBank acc. no. NM_{—}020363) (Table 13), that amplified a 120 bp fragment from genomic DNA of man, to generate a probe for screening the testis cDNA library and the bovine RPCI-42 BAC library (BAC/PAC Resources Center, Calif.) for the bovine DAZL. The PCR products were labeled with isotope 32P, and used as a probe to screen the male bovine BAC library. The PCR products amplified from the positive BAC clones were sequenced at the Advanced Genetic Analysis Center (AGAC), University of Minnesota (UMN), to confirm the presence of gene sequences in the positive clones.

Bovine testis cDNA library construction and screening Total RNA was extracted from an adult testis of a healthy bull (Chomczynski and Sacchi, 1987), and mRNA was isolated with the Poly A Tract Isolation System (Promega, Madison, Wis.) according to the manufacturer's protocol. In order to obtain a library with larger inserts and higher titer, three cDNA libraries (S/L1, S/L3, and E/L3) were constructed with different ratios of vector-to-cDNA insert during ligation, using the ZAP Express cDNA Synthesis Kit (Stratagene, La. Jolla, Calif.). The E/L3 library was screened with the human DAZ2 probe. Positive clones were isolated and the phagemid DNA was purified by a Miniprep Kit (Perkin Elmer, Foster City, Calif.) and sequenced at AGAC, UMN.

Fluorescence in situ Hybridization (FISH)

The BAC DNAs from clones 304I5, 94B4, 162L5 and 248K19 that were positive for the DAZ2 probe were labeled with biotin-14-dATP using the GIBCO BRL BioNick™ labeling kit. Metaphase chromosome slides were prepared using standard procedures from peripheral blood lymphocytes obtained from a normal, healthy bull, and FISH experiments were carried out essentially as described (Liu and Ponce de Leon, 2004).

DAZL Genomic Structure Analysis

The genomic structure of bDAZL was analyzed using a PCR strategy in combination with a BLAST search approach against the bovine genome sequence (Build 2.1) (http://www.ncbi.nlm.nih.gov/genome/seq/BtaBlast.html). Primers flanking the possible introns were designed from the DAZL cDNA sequence using PRIMER3 (http://frodo.wi.mit.edu/cqi-bin/primer3/primer3_www.cgi) (Rozen and Skaletsky, 2000) (Table 13). PCR with different combinations of primer pairs was performed in a 96-well Techne Touchgene thermocycler using the DAZL positive BAC clone or genomic DNA as templates with the DAZL cDNA clone and the testis cDNA libraries as positive controls. Each PCR reaction contained 20 ng of DNA, 1× buffer (TaKaRa, Biomedicals), 2 mM MgCl2, 200 μM dNTPs, 5 μmol of each primer and 0.5 U TaKaRa Ex Taq DNA polymerase in a total volume of 20 μl. The amplification cycle included denaturation at 95° C. for 5 min, followed by 36 cycles at 94° C. for 1 min, 52-64° C. for 1 min, 72° C. for 1-7 min depending upon the intron size, and a final extension step at 72° C. for 7 min. PCR products containing any intron sequences were sequenced at AGAC, UMN.

RT-PCR

Total RNA was extracted from 13 different bovine tissues, including testis, ovary, lung, liver, kidney, spleen, cerebellum, adrenal, longissimus, lymphnode, thymus, semitendon and peripheral blood mononuclear cells (PBMC), using Trizol reagent (Gibco/Life Technologies, Grand Island, N.Y.) as per the manufacturer's instruction. The purity and integrity of the RNA samples were verified as described (Pelle and Murphy, 1993). For RT-PCR analysis, 1 to 5 μg of total RNA in 10 μl of RNase-free water was incubated at 70° C. for 10 min in the presence of 1 μg of random hexamers or oligo(dT) 15 (Invitrogen, Carlsbad, Calif.) and 2.5 mM of each dNTP. The reaction sample was chilled immediately and the following components were added for a final volume of 20 μl: 5×first strand buffer, 0.1 mM dithiothreitol (DTT), 40 U of RNase inhibitor and 200 U of Superscript IIIRNase H—reverse transcriptase (Invitrogen, Carlsbad, Calif.). The reaction was incubated at 50° C. for 50 min and finally heated at 85° C. for 5 min to deactivate the transcriptase activity. Two μl aliquots from the first-strand cDNA synthesis reaction were then amplified in a 50 μl reaction containing 1×PCR buffer, 1.5 mM MgCl2, 200 μM dNTPs, 5 μmol of each primer (DAZL416F and DAZL621R) and 2.5 U of Taq polymerase. The amplification cycle included an initial 95° C., 7 min denaturing step, followed by 35 cycles consisting of 94° C. for 1 min, 60° C. for 1 min, and 72° C. for 1 min, and a final extension step at 72° C. for 7 min. The PCR products were analyzed by electrophoresis in a 1.5% agarose gel stained with ethidium bromide.

TABLE 13

Sequences of oligonucleotide
primers used for PCR and RT-PCR

| | |
|---|---|
| Primer name | Sequence |

| | |
|---|---|
| DAZ2Fab | 5′-CTCAACCATCTCCAGAGAGG-3′ |
| DAZ2Rac | 5′-CTAGCATCAATTCCACCAAC-3′ |
| DAZL54Fd | 5′-GAGCAGACCCAACAGCAG-3′ |
| DAZL71F | 5′-CTGCTGTTGGGTCTGCTC-3′ |
| DAZL118F | 5′-CCTCTGAGTGAACACTGG-3′ |
| DAZL136F | 5′-CTGTCGCCATCATGTCTGC-3′ |
| DAZL231F | 5′-GGCTATGTTTTACCAGAAGG-3′ |
| DAZL250R | 5′-CCTTCTGGTAAAACATAGCC-3′ |
| DAZL382R | 5′-GACACACCAGTTCGATCC-3′ |
| DAZL416F | 5′-CGTGGATGTGCAGAAGATAG-3′ |
| DAZL433R | 5′-ATCTTCTGCACATCCACGTC-3′ |
| DAZL487R | 5′-ATTGCAGGGCCCAGTTTCAG-3′ |
| DAZL602F | 5′-GCAGCCTCCAACCATGATAA-3′ |
| DAZL621R | 5′-TTATCATGGTTGGAGGCTGC-3′ |
| DAZL817R | 5′-GGTAGAAGAGACAAAATCCTGAAC-3′ |
| DAZL925F | 5′-TTAATCCAGAGAATAGACTGAGAAAC-3′ |
| DAZL925R | 5′-GTTTCTCAGTCTATTCTCTGGATTAA-3′ |
| DAZL1008F | 5′-AGTCGGGCAGTGCTTAAGTC-3′ |
| DAZL1055F | 5′-GTCTCATAGGAAGTCACAGG-3′ |
| DAZL1074R | 5′-CCTGTGACTTCCTATGAGAC-3′ |
| DAZL1214F | 5′-GGTAGAAGAGACAAAATCCTGAAC-3′ |
| DAZL1237R | 5′-GTTCAGGATTTTGTCTCTTCTACC-3′ |
| DAZL1307R | 5′-GCAAAGACAGTATCAGCATAGG-3′ |
| GAPDH-Fe | 5′-GTGACACTCACTCTTCTACCTTTGATC-3′ |
| GAPDH-R | 5′-GGTCCACCACCCTGTTGCT-3′ |

a Primers were designed from the human DAZ2 mRNA (NM_020363), start position nt 226 for DAZ2F, and nt 345 for DAZ2R.
b F-forward primer.
c R-reverse primer.
d DAZL primer series were designed from the bovine DAZL cDNA isolated in this study. The numeral numbers after DAZL stand for the position in the cDNA.
e Primers designed from the mouse Gapdh mRNA, and amplified a 116 bp fragment.

DAZL gene

| | | Start-end | Start-end position | |
|---|---|---|---|---|
| | | position in | in the genomic | |
| Exon | Intron | the cDNA | DNAₐ | Size (bp) |
| 1 | | 1-149 | unknown | 149 |
| | 1 | | 88,265_{b}-69,473 | >18,793_{b} |
| 2 | | 150-296 | 69,472-69,326 | 147 |
| | 2 | | 69,325-68,911 | 415 |
| 3 | | 297-388 | 68,910-68,819 | 92 |
| | 3 | | 68,818-68,243 | 576 |
| 4 | | 389-440 | 68,242-68,191 | 52 |
| | 4 | | 68,190-67,746 | 445 |
| 5 | | 441-504 | 67,745-67,682 | 64 |
| | 5 | | 67,681-67,590 | 92 |
| 6 | | 505-644 | 67,589-67,450 | 140 |
| | 6 | | 67,449-64,353 | 3,097 |
| 7 | | 645-716 | 64,352-64,281 | 72 |
| | 7 | | 64,280-63,701 | 580 |
| 8 | | 717-767 | 63,700-63,650 | 51 |
| | 8 | | 63,649-62,848 | 802 |
| 9 | | 768-881 | 62,847-62,734 | 114 |
| | 9 | | 62,733-61,636 | 1,098 |
| 10 | | 882-980 | 61,635-61,537 | 99 |
| | 10 | | 61,536-56,678 | 4,859 |
| 11 | | 981-1,331 | 56,677-54,704 | 1,974_{c} |

ₐThe Bos taurus chromosome Un genomic contig (BtUn_WGA7606).
_{b}As the 5′ UTR in the cDNA is not present in the region of 88,265-69,473 in the genomic contig, it means the start position of the intron 1 is excluded from the contig, indicating that the intron 1 is larger that 18,793 bp.
_{c}The 1,623 bp is included.

### Results and Discussion

Cloning and sequence analysis of bDAZL. We screened the bovine testis cDNA library E/L3 with the human DAZ2 PCR probe and isolated four positive cDNA clones. One of the positive clones, namely 11.1A.3, was selected for further analysis. Sequencing of this cDNA clone indicated that it contains a full-length mRNA that is 1,373 bp long (GenBank acc.no. DQ408765, FIG. 4). BLAST search against the NCBI database http://www.ncbi.nlm.nih.gov/BLAST/) showed that it is the bovine homolog of the human DAZ/DAZL and mouse Dazl gene. The cDNA includes a 146 bp 5′ untranslated region (UTR), an open reading frame (ORF) for a protein of 295 aa residues with an RNA recognition motif (RRM), and a 339 bp 3′ UTR FIG. 4). Compared to the human DAZ/DAZL sequences, the bDAZL coding region is closer to human DAZL (90% identical) than the DAZ cluster (84-89%). The latter contains 7-15 copies of a tandem repeat of 24 aa (72 bp) (so-called DAZ repeat) in its coding region. Like human DAZL, bDAZL contains only one DAZ repeat at position of nt 645-716 (FIG. 4). The bDAZL cDNA also has a high similarity to the mouse Dazl (91%). The sequence alignment further revealed that there is a breakpoint at nt 1,083 in the 3′ UTR of bDAZL, where a 1,577 bp sequence (nt 1,228-2,805 of NM_{—}001351) is present in the human DAZL 3′ UTR. A similar size fragment of 1,589 bp (nt 1,119-2,708 of NM_{—}010021) is also present in the mouse Dazl 3′ UTR. The deduced protein sequence of bDAZL contains 295 aa, identical in length to the human DAZL protein (NP_{—}001342), but three aa shorter than that of the mouse (NP_{—}034151). The peptide sequence of bDAZL is 97% similar to mouse Dazl, while 96% similar to human DAZL. The RRM region is located at aa 32-117 (FIG. 4), which is completely conserved between bovine and mouse. One amino acid substitution (Y to F) at the position of aa 88 is found in the RRM when compared to human DAZL (FIG. 4).

There are several entries in the GenBank for bDAZL, including a gene locus (LOC530116), an mRNA (XM_{—}608581) and a protein (XP_{—}608581.2), all predicted from the recently released bovine genome sequence (Build 2.1). When we compared the full length cDNA and the deduced aa sequence obtained in this study, we found that the predicted mRNA (XM_{—}608581) is a partial mRNA, and the predicted protein (XP_{—}608581.2) is different from the actual protein sequences of human, mouse and bovine DAZL in both the N- and C-terminals.

Alternative RNA splicing is found in the bDAZL 3′ UTR. To address questions as to why the mRNA of the bDAZL gene is much shorter than that of the human and mouse, and whether alternative splicing is responsible for the absence of the ˜1.6 kb sequence in the bDAZL 3′ UTR, we performed PCRs with DNA templates from the bovine genomic DNA, the bovine testis cDNA libraries and the DAZL cDNA clone using primer pair DAZL1055F/1237R (Table 13). This primer pair, flanking the breakpoint (nt 1,083), is predicted to amplify a 183 bp product from all DNA templates examined. When PCR products were separated on a 1% agarose gel (not shown), the primer pair was shown to amplify the predicted product from the cDNA clone. However, the primer amplified an ˜1.8 kb fragment instead of a 183 bp fragment from genomic DNA (both BAC DNA and male bovine genomic DNA). The primer pair amplified both fragments from the three testis cDNA libraries, indicating that there are two types of transcripts for bDAZL, which differ from each other in the 3′ UTR. RT-PCR with the same primer pair on the testis RNA sample isolated from another bull confirmed the presence of the two types of transcripts. The ratio of the 1.8 kb to the 183 bp products varied among the three testis cDNA libraries. The E/L3 library had the highest amount of transcripts for the small fragment, so did the RT-PCR products, which suggests that the shorter transcript predominated. Sequencing the PCR products indicated that the small fragment corresponded to the cDNA sequence, while the large fragment contained an additional 1,623 bp insertion in the position nt 1,083 of the cDNA (FIG. 4). The inserted sequences are 88 and 84% identical to the corresponding region in the human and mouse DAZL 3′ UTR, respectively. Here we refer to the longer transcript (2,996 bp) as bDAZL transcript variant 1 (GenBank acc. no. DQ408764), and the shorter transcript (1,373 bp) as bDAZL transcript variant 2 (GenBank acc. no. DQ408765). As the DAZL1055F/1237R primer pair amplified only the 1.8 kb fragment corresponding to variant 1 from bovine genomic DNA, we presume that only one copy of the bDAZL gene is active in the bovine genome. Taken together, these observations strongly suggest that the two transcripts are probably the products of alternative RNA splicing of the bDAZL gene.

A report that two DAZL transcripts, ˜3.5 kb and ˜2.3 kb, are expressed at different levels in the testis of cynomologus monkey (Carani et al., 1997) suggested that the ˜3.5 kb is the predominant transcript. It is not clear from the report which region the two transcripts share and which region is missing from the short version. Transcript variants are also found in the testis for the human DAZ cluster. For example, DAZ2 has three variants (Ferlin et al., 2004), which vary in the DAZ repeats of the coding region. The finding of the transcript variants in the 3′ UTR of bDAZL in the present study is important from a gene regulatory point of view. Many mRNAs undergo translational regulation, normally mediated by sequences within the UTRs.

Previous studies on the zebra fish DAZL (zDAZL) gene identified a sequence motif ‘GUUC’ in the zDAZL 3′ UTR that can activate the translation of its own mRNA (Maegawa et al., 2002). We compared the sequences of 3′ UTR from both variants to determine whether such a motif is present, and if there is a difference in copy number between the two bDAZL transcript variants. We found one ‘GUUC’ motif (starting at nt 2,899 in FIG. 4) common to both transcripts and four additional ‘GUUC’ motifs (nt 1,228; 2,247; 2,537; and 2,626; FIG. 4) within the 1,623 bp fragment unique to the transcript variant 1. According to the results of Maegawa et al. (2002), the level of DAZL translational activation is directly proportional to the copy number of ‘GUUC’ motifs. As such, bDAZL transcript variant 1 may be more active than the transcript variant 2 in protein translation.

Genomic organization of the bDAZL gene. Initially, a PCR strategy was used to analyze the genomic structure of the bDAZL gene. We used different combinations of PCR primers (Table 13) to amplify the gene fragments using DAZL positive BAC clones, male bovine genomic DNA, the DAZL cDNA and bovine testis cDNA libraries (as control). We sequenced all PCR products that amplified from the genomic DNA and were considered to contain intron sequence. We were able to identify the positions and sizes of exons 1 to 5 and introns 2 to 5 (Table 14, FIG. 5). By sequencing the BAC 30415 using primer DAZL118F and DAZL231R, we determined that intron 1 is inserted after the start codon at nt 149 of the bovine cDNA. As intron 1 is large, routine and long-PCR with primer pair DAZL118F/231R failed to determine the intron size.

To completely determine the genomic structure of the bDAZL gene, we then BLAST searched the bovine genome sequence (Build 2.1) (http://www.ncbi.nim.nih.gov/genome/seq/BtaBlast.html) using the bDAZL cDNA. The search yielded two sequences, one of which was a Bos taurus chromosome Un genomic contig (BtUn_WGA7606), with an E-value of 3e-136. Multiple sequence alignment among the BtUn_WGA7606, the DAZL cDNA and the sequence contig for the exons 1-5 and introns 2-5 generated by PCR in this study indicated that this genomic contig contains the bDAZL gene except for the 5′ UTR and exon 1 region. It confirmed our PCR mapping results for the exons 2-5 and intron 2-5 at a 100% identity and further revealed the existence of additional exons (6-11) and introns (6-10) in the bDAZL gene (Table 14, FIG. 5A). It also confirmed that the additional 1,623 bp in the transcript variant 1, spliced out from the transcript variant 2 at nt 1,083 of the bDAZL cDNA, is present in the genomic contig (nt 54,951-56,573 bp) (FIG. 4, FIG. 5 A). The results demonstrated that the bDAZL gene structure is very similar to human DAZL and is composed of 11 exons and 10 introns (Suppl. Table 2, Suppl. FIG. 2). Exon size ranges from 51 to 147 bp, while the size of introns 2-10 ranges from 92 to 4,859 bp (Table 14). Even with this genomic contig, the precise size of intron 1 still could not be determined. Since the 5′ UTR plus the start codon (nt 1-149) in the bovine cDNA do not match any sequence in the nt 69,473-88,265 region in the genomic contig BtUn_WGA7606, we estimated that the intron 1 in bDAZL is larger than 18,793 bp. Therefore, the bDAZL gene spans at least 33 kb in the genome (FIG. 5A). By comparison, both human (FIG. 5B) and mouse DAZL have 11 exons and 10 introns, and span ˜19 and 14 kb, respectively. Furthermore, as the genomic contig BtUn_WGA7606 does not contain the entire bDAZL gene (FIGS. 5 A and B), the incorrect prediction of the bDAZL mRNA and protein in the bovine genome sequence project is anticipated.

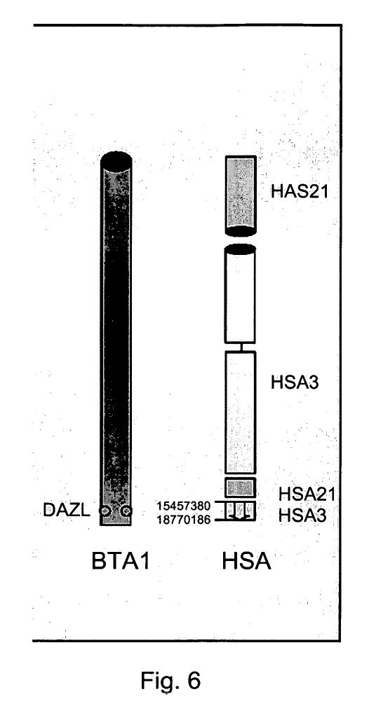

bDAZL maps to BTA1q. In order to map bDAZL, we screened the male bovine BAC library (RPCI-42) with the DAZL probe and isolated four positive BAC clones. BAC finger printing analysis indicated that the four positive BACs overlapped forming a contig (data not shown). All four BAC clones were labeled with Biotin dUTP and FISH mapped on bovine male metaphase chromosome spreads. Strong and distinct hybridization signals were observed at the distal region of the long arm of BTA1 for all four BAC probes (data not shown). No signal was observed on other chromosomes including the X and Y (FIG. 6). In the human, besides the four copies of DAZ on the Y chromosome, the autosomal gene DAZL maps on HSA3 (3p24.3) at nt 16,622,010-16,603,307 (Build 35, FIG. 6). The recently published cattle-human whole genome comparative maps (Everts-van der Wind et al., 2005; Itoh et al., 2005) demonstrated that BTA1 is conserved with segments of HSA21 and HSA3, including the HSA3 sequence of nt 15,457,380-18,770,186 homologous to the distal region of BTA1q (FIG. 6), in agreement with the FISH results, mapping bDAZL to BTA1q.

Mapping this gene to an autosome in cattle further confirms previous work that all mammals except for humans, great apes and Old World monkeys, do not have a DAZ gene on their Y-chromosomes, but instead have a DAZL gene on an autosome (Saxena et al., 1996; Skaletsky et al., 2003). In bovine genome sequence Build 2.1, the unmapped sequence contig BtUn_WGA7606 was not associated with any BAC clone. Mapping of the bDAZL gene to this genomic contig in the present study provides direct evidence for placing this contig at the distal region of BTA1q in future assemblies of the bovine genome sequence. Furthermore, four BAC clones were isolated for the DAZL gene from the bovine RPCI-42 BAC library. BAC fingerprint analysis indicated that these BACs form a contig that spans about 300 kb. Comparing the BAC contig with the genomic contig BtUn_WGA7606 in future studies will allow identification of any overlapped regions. Sequencing the non-overlapped region(s) from the BAC contig will serve two purposes. First, determine the size of intron 1 for the DAZL gene; second, close the gap for BTA1 in that region. Even if the BAC contig is not large enough to close the gap, it will provide a starting point for closure of that gap in future sequence assembly.

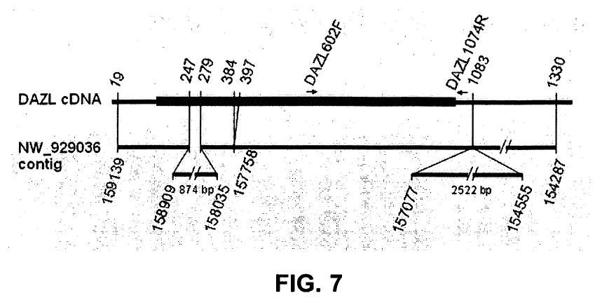

A DAZL pseudogene is present on BTA16. During the analysis of the bDAZL genomic structure, primer pair DAZL602F/1074R amplified a 473 bp fragment (FIG. 7) across all DNA samples examined. When this fragment was sequenced, we found that the sequence amplified from the genomic DNA was only 87% identical to the corresponding sequence amplified from the cDNA clone and cDNA libraries, raising the question whether there are two copies of the DAZL gene in the bovine genome. As mentioned above, the BLAST search against the bovine genome sequence (Build 2.1) with the bDAZL cDNA resulted in two hits: one was the genomic contig (BtUn_WGA7606), described above, that contains the DAZL gene, the other was a Bos taurus chromosome 16 genomic contig (Bt16_WGA2706). Surprisingly, the latter had a lower E-value of 0.0 with a score of 652, suggesting that this genomic contig most likely contains a copy of the bDAZL gene. Further sequence comparison showed that the BTA16 genomic contig does contain a segment that is homologous to the entire bDAZL cDNA (FIG. 7). The overall sequence identity between the two sequences is ˜87%. While a 13 bp (nt 384-397 in the bDAZL cDNA) fragment is deleted from the NW_{—}929036 genomic sequence at position nt 157,758, two large insertions are found in the genomic sequence (FIG. 7). The first insertion is 874 bp (nt 158,035-158,909) long, referring to a position between nt 247 and 279 in the cDNA. The second insertion is 2,522 bp (nt 154,555-157,077), which is inserted exactly at the position of the break point (nt 1,083) in the cDNA (FIG. 7). Although the 2,522 bp fragment is 899 bp larger than the 1,623 bp fragment obtained from the DAZL transcript variant 1, the two sequences show 78-91% identity from region to region, although several gaps exist. The deletion and the first insertion occur in locations or responding to the RRM region in DAZL (FIG. 4), probably leading to a loss of function. These results indicated that the DAZL homologous sequence in BTA16 is almost certainly a duplicate of the DAZL cDNA, originated through a reverse transcription mechanism.

Bovine DAZL gene is expressed in testis only. Expression of bDAZL mRNA was analyzed by RT-PCR in 13 different tissues including both male and female gonads. Primer pair DAZL416F/621R (Table 13) flanking exon 4 and exon 6 was expected to amplify a 206 bp fragment from the RNA samples and a 743 bp from genomic DNA. A housekeeping gene glyceraldehyde 3-phosphate dehydrogenase (GAPDH) was used as an internal control. Ti was observed that while the GAPDH is expressed across all tissues analyzed, the expected 206 bp RTPCR product was amplified only from the testis RNA and the three testis cDNA libraries, indicating that the bDAZL gene is expressed in testis only.

The spatiotemporal expression of the DAZL gene varies among species investigated. Human DAZL, like the DAZ cluster, was found to be testis-specific (Yen et al., 1996), but later was found expressed in the ovary (Seligman and Page, 1998; Dorfman et al., 1999; Cauffman et al., 2005) and in PGCs in fetal gonads (Xu et al., 2001). Mouse Dazl is also expressed in both male and female gonads (Cooke et al., 1996). In the cynomologus monkey, DAZL was considered to be expressed only in the testis (Carani et al., 1997). However, transcripts from the ovary were not included in the Northern blot analysis (Carani et al., 1997). In the present study, RT-PCR revealed that bDAZL is expressed only in the testis.

In conclusion, we report the cloning of the bDAZL cDNA and the deduced aa sequences, which revise the prediction from the bovine genome sequence (build 2.1). We further demonstrated that DAZL is expressed only in the bovine testis and maps to the distal region on BTA1q. Additional bDAZL is highly conserved with the human and mouse DAZL in mRNA, protein and even in the genomic organization levels.

### REFERENCES

Agulnik A I, Zharkikh A, Boettger-Tong H, Bourgeron T, McElreavey K and Bishop C E (1998) Evolution of the DAZ gene family suggests that Y-linked DAZ plays little, or a limited, role in spermatogenesis but underlines a recent African origin for human populations. Hum Mol Genet 7(9):1371-1377. Burgoyne P S (1996) Fruit(less) flies provide a clue. Nature 381(6585):740 741.
Cauffman G, Van de Velde H, Liebaers I and Van Steirteghem A (2005) DAZL expression in human oocytes, preimplantation embryos and embryonic stem cells. Mol Hum Reprod 11 (6):405-411.
Cooke H J, Lee M, Kerr S and Ruggiu M (1996) A murine homologue of the human DAZ gene is autosomal and expressed only in male and female gonads. Hum Mol Genet. 5(4):513-516.
Eberhart C G, Maines J Z and Wasserman S A (1996) Meiotic cell cycle requirement for a fly homologue of human Deleted in Azoospermia. Nature 381 (6585):783-785.
Ferlin A, Moro E, Garolla A and Foresta C (1999) Human male infertility and Y chromosome deletions: role of the AZF-candidate genes DAZ, RBM and DFFRY. Hum Reprod 14(7):1710-1716.
Gromoll J, Weinbauer G F, Skaletsky H, Schlatt S, Rocchietti-March M, Page D C and Nieschlag E (1999) The Old World monkey DAZ (Deleted in AZoospermia) gene yields insights into the evolution of the DAZ gene cluster on the human Y chromosome. Hum Mol Genet. 8(11):2017-2024.
Houston D W and King M L (2000) A critical role for Xdazl, a germ plasm-localized RNA, in the differentiation of primordial germ cells in Xenopus. Development 127(3):447-456.
Lin Y M, Chen C W, Sun H S, Tsai S J, Hsu C C, Teng Y N, Lin J S and Kuo P L (2001) Expression patterns and transcript concentrations of the autosomal DAZL gene in testes of azoospermic men. Mol Hum Reprod 7(11): 1015-1022.
Maegawa S, Yamashita M, Yasuda K and Inoue K (2002) Zebrafish DAZ-like protein controls translation via the sequence ‘GUUC’. Genes Cells 7(9):971-984.
Reijo R, Lee T Y, Salo P, Alagappan R, Brown L G, Rosenberg M, Rozen S, Jaffe T, Straus D, Hovatta O and et al. (1995) Diverse spermatogenic defects in humans caused by Y chromosome deletions encompassing a novel RNA-binding protein gene. Nat Genet. 10(4):383-393.
Ruggiu M, Speed R, Taggart M, McKay S J, Kilanowski F, Saunders P, Dorin J and Cooke H J (1997) The mouse Dazla gene encodes a cytoplasmic protein essential for gametogenesis. Nature 389(6646):73-77.
Saxena R, Brown L G, Hawkins T, Alagappan R K, Skaletsky H, Reeve M P, Reijo R, Rozen S, Dinulos M B, Disteche C M and Page D C (1996) The DAZ gene cluster on the human Y chromosome arose from an autosomal gene that was transposed, repeatedly amplified and pruned. Nat Genet. 14(3):292-299.
Shan Z, Hirschmann P, Seebacher T, Edelmann A, Jauch A, Morell J, Urbitsch P and Vogt P H (1996) A SPGY copy homologous to the mouse gene Dazla and the Drosophila gene boule is autosomal and expressed only in the human male gonad. Hum Mol Genet. 5(12):2005-2011.
Slee R, Grimes B, Speed R M, Taggart M, Maguire S M, Ross A, McGill N I, Saunders P T and Cooke H J (1999) A human DAZ transgene confers partial rescue of the mouse Dazl null phenotype. Proc Natl Acad Sci USA 96(14):8040-8045.
Teng Y N, Lin Y M, Lin Y H, Tsao S Y, Hsu C C, Lin S J, Tsai W C and Kuo P L (2002) Association of a single-nucleotide polymorphism of the deleted-in-azoospermia-like gene with susceptibility to spermatogenic failure. J Clin Endocrinol Metab 87(11):5258-5264.
Tung J Y Rosen M P, Nelson L M, Turek P J, Witte J S, Cramer D W, Cedars M I, Pera R A. Variants in Deleted in AZoospermia-Like (DAZL) are correlated with reproductive parameters in men and women. (2006) Hum Genet. 118:730-740.
Xu E Y, Moore F L and Pera R A (2001) A gene family required for human germ cell development evolved from an ancient meiotic gene conserved in metazoans. Proc Natl Acad Sci USA 98(13):7414-7419.
H. P. Donald and J. L. Hancock. (1953) Evidence of a gene controlled sterility in bulls. J. Agric. Sci. 43:178-181.
Kovacs A, Villagomez DA, Gustavsson I, Lindblad K, Foote R H, Howard T H. (1992) Synaptonemal complex analysis of a three-breakpoint translocation in a subfertile bull. Cytogenet Cell Genet. 61(3):195-201.
Ansari H A, Jung H R, Hediger R, Fries R, Konig H, Stranzinger G. (1993) A balanced autosomal reciprocal translocation in an azoospermic bull. Cytogenet Cell Genet. 62(2-3):117-23.
Villagomez D A, Andersson M, Gustavsson I, Ploen L. (1993) Synaptonemal complex analysis of a reciprocal translocation, rcp(20; 24) (q17; q25), in a subfertile bull. Cytogenet Cell Genet. 62(2-3):124-30.
Iannuzzi L, Di Meo G P, Leifsson P S, Eggen A, Christensen K. (2001) A case of trisomy 28 in cattle revealed by both banding and FISH-mapping techniques. Hereditas. 134(2):147-51.
Moura A A, Erickson B H. (2001) Testicular development, histology, and hormone profiles in three yearling angus bulls with spermatogenic arrest. Theriogenology. 55(7):1469-88.
Parkinson T J. (2000) Nuclear vacuolation as a cause of sterility in an angus bull. Vet J. 159(2):207-10.
Steffen D. (1997) Genetic causes of bull infertility. Vet Clin North Am Food Anim Pract. 13(2):243-53.
Yang X J, Shinka T, Nozawa S, Yan H T, Yoshiike M, Umeno M, Sato Y, Chen G, Iwamoto T, Nakahori Y “Survey of the two polymorphisms in DAZL, an autosomal candidate for the azoospermic factor, in Japanese infertile men and implications for male infertility” Mol Hum Reprod. 2005 July; 11(7):513-5.
Thangaraj K, Deepa S R, Pavani K, Gupta N J, Reddy P, Reddy A G, Chakravarty B N, Singh L. “A to G transitions at 260, 386 and 437 in DAZL gene are not associated with spermatogenic failure in Indian population” Int J Androl. 2006 October; 29(5):510-14.
L. Bartoloni, C. Cazzadore, A. Ferlin, A. Garolla and C. Forestal “Lack of the T54A polymorphism of the DAZL gene in infertile Italian patients” Molecular Human Reproduction Vol. 10, No. 8 pp. 613-615, 2004; Advance Access publication Jun. 25, 2004.
Becherini L, Guarducci E, Degl'lnnocenti S, Rotondi M, Forti G, Krausz C “DAZL polymorphisms and susceptibility to spermatogenic failure: an example of remarkable ethnic differences” Int J Androl. 2004 December; 27(6):375-81.
Tschanter P, Kostova E, Luetjens C M, Cooper T G, Nieschlag E, Gromoll J. No association of the A260G and A386G DAZL single nucleotide polymorphisms with male infertility in a Caucasian population. Hum Reprod. 2004 December; 19(12):2771-6. Epub 2004 Nov 1.
Teng Y N, Lin Y M, Sun H F, Hsu P Y, Chung C L, Kuo P L. Association of DAZL haplotypes with spermatogenic failure in infertile men. Fertil Steril. 2006 July; 86(1):129-35. Epub 2006 May 30.
Tung J. Y., Rosen, M. P, Nelson L. M., Turek P. J., Witte J. S., Cramer D. W., Cedars M. I., and Reijo-Pera R. A., Novel missense mutations of the Deleted-in-AZoospermia-Like (DAZL) gene in infertile women and men, Reprod Biol Endocrinol. 2006; 4: 40, Published online 2006 Aug. 2.
Carani C, Gromoll J, Brinkworth M H, Simoni M, Weinbauer G F, Nieschlag E: cynDAZLA:a cynomolgus monkey homologue of the human autosomal DAZ gene. Mol Hum Reprod 3:479-483 (1997).
Chomczynski P, Sacchi N: Single-step method of RNA isolation by acid guanidinium thiocyanate-phenol-chloroform extraction. Anal Biochem 162:156-159 (1987).
Dorfman D M, Genest D R, Reijo Pera R A: Human DAZL1 encodes a candidate fertility factor in women that localizes to the prenatal and postnatal germ cells. Hum Reprod 14:2531-2536 (1999).
Everts-van der Wind A, Larkin D M, Green C A, Elliott J S, Olmstead C A, et al: A high-resolution whole-genome cattle-human comparative map reveals details of mammalian chromosome evolution. Proc Natl Acad Sci USA 102:18526-18531 (2005).
Ferlin A, Bettella A, Tessari A, Salata E, Dallapiccola B, Foresta C: Analysis of the DAZ gene family in cryptorchidism and idiopathic male infertility. Fertil Steril 81:1013-1018 (2004).
Itoh T, Watanabe T, Ihara N, Mariani P, Beattie C W, et al: A comprehensive radiation hybrid map of the bovine genome comprising 5593 loci. Genomics 85:413-424 (2005).
Liu W S, Ponce de Leon F A: Assignment of S R, ANT3, and CSF2RA to the bovine Ychromosome by FISH and RH mapping. Anim Biotechnol 15:103-109 (2004).
Pelle R, Murphy N B: Northern hybridization: rapid and simple electrophoretic conditions Nucleic Acids Res 21:2783-2784 (1993).
Rozen S, Skaletsky H: Primer3 on the WWW for general users and for biologist programmers. Methods Mol Biol 132:365-386 (2000).
Seligman J, Page D C: The Dazh gene is expressed in male and female embryonicgonads before germ cell sex differentiation. Biochem Biophys Res Commun 245:878-882 (1998).
Skaletsky H, Kuroda-Kawaguchi T, Minx P J, Cordum H S, Hillier L, et al: The malespecific region of the human Y chromosome is a mosaic of discrete sequence classes. Nature 423:825-837 (2003).
Yen P H: Putative biological functions of the DAZ family. Int J Androl 27:125-129 (2004).
Yen P H, Chai N N, Salido E C: The human autosomal gene DAZLA: testis specificity and a candidate for male infertility. Hum Mol Genet. 5:2013-2017 (1996).

1. A method for assessing the fertility of bovine males, comprising
(a) obtaining a sample of genetic material from an individual male bovine;
(b) assaying in a DAZL gene of that genetic material for the presence of a particular allele of single nucleotide polymorphisms (SNPs) that forms a unique haplotype in the individual male bovine that is associated with decreased fertility thereby identifying that individual male bovine as one that will not exhibit a higher rate of successful impregnation; and/or
(c) assaying in a DAZL gene of the genetic material for the presence of a particular allele of single nucleotide polymorphisms (SNPs) that forms a unique haplotype in the individual male bovine that is associated with enhanced fertility thereby identifying that individual male bovine as one that will exhibit a higher rate of successful impregnation.

2. The method of claim 1 wherein the higher rate of successful impregnation is assessed during dairy and beef cattle reproduction.

3. The method of claim 1 wherein the male bovine is a bovine being considered for use in dairy cattle reproduction.

4. The method of claim 1 wherein the genotype of the male bovine and its associated haplotypes is characterized by one or more SNPs in the introns, exons, the 5′UTR and 3′UTR regions of the bDAZL gene.

5. The method of claim 1 wherein the genetic material is obtained from a male bovine at or soon after birth (days or weeks).

6. The method of claim 1 wherein the genetic material is obtained in utero.

7. The method of claim 1 wherein the higher rate of successful impregnation is measured as a higher than average non-return rate.

8. The method of claim 1 wherein the unique haplotype is a haplotype identified in Table 7, 9 or 11 herein.

9. The method of claim 1 wherein the SNPs that form a unique haplotype comprising two or more of the SNPs of the bDAZL gene.

10. The method of claim 1 wherein the haplotype is characterized by one or more SNPs in the introns or the 3′ UTR region of the gene.

11. The method of claim 1 wherein the haplotype does not comprise an SNP associated with a change in an amino acid in the protein expressed by the gene.

12. The method of claim 1 wherein the haplotype is characterized by the sequence AACAATAACT.

13. The method of claim 1 further comprising a step of genetically identifying potentially infertile males in the population of male bovines being assessed by assaying for the presence of a deletion or a change in amino acid in the coding sequence of the bDAZL gene.

14. The method of claim 1 wherein the haplotype is characterized by three, four, five, six, seven, eight, nine or ten SNPs of the bDAZL gene.

15. The method of claim 1 wherein the haplotype consists essentially of three, four, five, six, seven, eight, nine or ten SNPs of the bDAZL gene.

16. A method for breeding cattle to obtain male offspring which will produce semen which will exhibit a higher rate of successfully impregnation by either artificial insemination or natural mating during dairy and beef cattle reproduction which comprises the steps of:
(a) obtaining a sample of genetic material from each of the male and female bovine individuals which are to be bred;
(b) assaying each sample for the presence of a particular allele of single nucleotide polymorphisms (SNPs) that forms a unique haplotype in the individual bovine that is associated with decreased fertility thereby identifying that individual bovine as one that is undesirable for breeding to generate higher fertility male offspring; and/or
(c) assaying for the presence of a particular allele of single nucleotide polymorphisms (SNPs) that forms a unique haplotype in the individual bovine that is associated with enhanced fertility thereby identifying that individual bovine as one that that is undesirable for breeding to generate higher fertility male offspring; and
(d) using the results obtained to select pairs of male and female animals for breeding.

17. The method of claim 16 wherein the haplotype is characterized by one or more SNPs in the introns, exons, the 5′UTR or 3′UTR regions of the bDAZLgene.

18. The method of claim 16 wherein the haplotype is a haplotype identified in Table 7, 9 or 11 herein.

19. The method of claim 16 wherein the haplotype comprises two or more of the SNPs of Table 2A herein.

20. The method of claim 16 wherein the haplotype consists essentially of three, four, five, six, seven, eight, nine or ten SNPs of bDAZL as described herein.

21-45. (canceled)
